SEQUENCE LISTING (45 sequences)

SEQ ID NO: 1 - protein, 885 aa, Piromyces sp. E2
MSGLQMFQNLSLYGSLAEIDTSEKLNEAMDKLTAAQEQFREYNQEQVDKIFKAVALAASQNRVAFAKYAHEETQKGVFEDKVIKNEFAADYIYHKYCNDKTAGIIEYDEANGLMEIAEPVGPVVGIAPVTNPTSTIIYKSLIALKTRNCIIFSPHPGAHKASVFVVKVLHQAAVKAGAPENCIQIIFPKMDLTTELLHHQKTRFIWATGGPGLVHASYTSGKPALGGGPGNAPALIDETCDMNEAVGSIVVSKTFDCGMICATENAVVVVESVYENFVATMKKRGAYFMTPEETKKASNLLFGEGMRLNAKAVGQTAKTLAEMAGFEVPENTVVLCGEASEVKFEEPMAHEKLTTILGIYKAKDFDDGVRLCKELVTFGGKGHTAVLYTNQNNKDRIEKYQNEVPAFHILVDMPSSLGCIGDMYNFRLAPALTITCGTMGGGSSSDNIGPKHLLNIKRVGMRRENMLWFKIPKSVYFKRAILSEALSDLRDTHKRAIIITDRTMTMLGQTDKIIKACEGHGMVCTVYDKVVPDPTIKCIMEGVNEMNVFKPDLAIALGGGSAMDAAKMMRLFYEYPDQDLQDIATRFVDIRKRVVGCPKLGRLIKTLVCIPTTSGTGAEVTPFAVVTSEEGRKYPLVDYELTPDMAIVDPEFAVGMPKRLTSWTGIDALTHAIESYVSIMATDFTRPYSLRAVGLIFESLSLAYNNGKDIEAREKMHNASAIAGMAFANAFLGCCHSVAHQLGSVYHIPHGLANALMLSHIIKYNATDSPVKMGTFPQYKYPQAMRHYAEIAELLLPPTQVVKMTDVDKVQYLIDRVEQLKADVGIPKSIKETGMVTEEDFFNKVDQVAIMAFDDQCTGANPRYPLVSELKQLMIDAWNGVVPKL

SEQ ID NO: 2 - protein, 683 aa, Saccharomyces cerevisiae
MTIKEHKVVYEAHNVKALKAPQHFYNSQPGKGYVTDMQHYQEMYQQSINEPEKFFDKMAKEYLHWDAPYTKVQSGSLNNGDVAWFLNGKLNASYNCVDRHAFANPDKPALIYEADDESDNKIITFGELLRKVSQIAGVLKSWGVKKGDTVAIYLPMIPEAVIAMLAVARIGAIHSVVFAGFSAGSLKDRVVDANSKVVITCDEGKRGGKTINTKKIVDEGLNGVDLVSRILVFQRTGTEGIPMKAGRDYWWHEEAAKQRTYLPPVSCDAEDPLFLLYTSGSTGSPKGVVHTTGGYLLGAALTTRYVFDIHPEDVLFTAGDVGWITGHTYALYGPLTLGTASIIFESTPAYPDYGRYWRIIQRHKATHFYVAPTALRLIKRVGEAEIAKYDTSSLRVLGSVGEPISPDLWEWYHEKVGNKNCVICDTMWQTESGSHLIAPLAGAVPTKPGSATVPFFGINACIIDPVTGVELEGNDVEGVLAVKSPWPSMARSVWNHHDRYMDTYLKPYPGHYFTGDGAGRDHDGYYWIRGRVDDVVNVSGHRLSTSEIEASISNHENVSEAAVVGIPDELTGQTVVAYVSLKDGYLQNNATEGDAEHITPDNLRRELILQVRGEIGPFASPKTIILVRDLPRTRSGKIMRRVLRKVASNEAEQLGDLTTLANPEVVPAIISAVENQFFSQKKK

SEQ ID NO: 3 - DNA, 2655 bp, Artificial Sequence
  Yeast codon-optimised sequence
atgtccggtttacaaatgttccaaaacttgtccttgtacggttccttggctgaaattgatacttccgaaaagttgaacgaagctatggataagttgactgctgctcaagaacaattcagagaatacaatcaagaacaagtcgacaagattttcaaggctgttgctttggctgcttctcaaaacagagttgcttttgctaaatacgcccatgaagaaactcaaaagggtgttttcgaagataaggtcattaagaacgaattcgctgccgattacatctaccataagtactgtaatgataagaccgccggtatcatcgaatacgatgaagctaatggtttgatggaaattgctgaaccagttggtccagttgttggtattgctccagttactaatccaacctctaccatcatctacaagtccttgattgctttgaaaaccagaaactgcatcatcttctcaccacatccaggtgctcataaggcttcagtttttgttgttaaggtcttgcatcaagctgctgttaaggctggtgctccagaaaactgtattcaaattatcttcccaaagatggacttgaccaccgaattattgcatcatcaaaagaccagattcatttgggctactggtggtccaggtttggttcatgcttcttatacttcaggtaaaccagctttgggtggtggtcctggtaatgctcctgctttgattgacgaaacttgtgatatgaatgaagccgttggttccatcgttgtttctaagacttttgattgcggtatgatttgcgctactgaaaacgccgttgttgttgtagaatctgtctacgaaaatttcgttgccactatgaagaaaagaggtgcttactttatgaccccagaagaaacaaagaaggcctctaatttgttgttcggtgaaggtatgagattgaacgctaaagctgttggtcaaactgctaaaactttagctgaaatggctggtttcgaagtcccagaaaacactgttgttttgtgtggtgaagcttccgaagttaagtttgaagaacctatggctcacgaaaaattgaccactattttgggtatctacaaggccaaggatttcgatgatggtgttagattgtgcaaagaattggttacttttggtggtaagggtcatactgctgtcttgtacactaatcaaaacaacaaggacagaatcgaaaagtaccaaaacgaagttccagccttccatatcttggttgatatgccatcttctttgggttgcattggtgatatgtacaactttagattggctccagctttgactattacctgtggtactatgggtggtggttcttcttctgataatatcggtccaaaacacttgttgaacatcaagagagttggtatgagaagagaaaacatgttatggttcaagatcccaaagtccgtctacttcaaaagagccattttgtctgaagctttgtccgatttgagagatactcataagagagccattatcatcaccgatagaactatgactatgttgggtcaaaccgacaagatcattaaggcttgtgaaggtcatggtatggtctgtactgtttacgataaggttgttccagatccaaccattaagtgcattatggaaggtgtcaacgaaatgaacgttttcaaaccagatttggctattgcattaggtggtggtagtgctatggatgctgctaaaatgatgagattattctacgaatacccagaccaagacttgcaagatattgctactagattcgtcgacatcagaaaaagagttgtcggttgtccaaaattgggtagattgattaagaccttggtctgtattccaactacttctggtactggtgctgaagttactccttttgctgttgttacttctgaagaaggtagaaagtacccattggttgattacgaattgactccagatatggcaatcgttgatccagaatttgccgttggtatgccaaaaagattgacttcttggactggtattgatgctttgacccatgctattgaatcctacgtttctattatggctaccgatttcaccagaccttattctttgagagcagttggtttgatcttcgaatctttgtctttggcctacaacaatggtaaggatatcgaagccagagaaaagatgcataacgcttcagctattgctggtatggcatttgctaatgcttttttgggttgttgccattccgttgctcatcaattgggttctgtttaccatattccacatggtttggcaaacgctttgatgttgtctcacattattaagtacaacgccactgattccccagttaagatgggtacatttccacaatacaaatacccacaagccatgagacattacgcagaaattgcagaattgttgttgcctccaactcaagttgttaagatgaccgatgttgataaggtccaatacttgatcgacagagtcgaacaattgaaagctgatgtcggtattccaaagtccatcaaagaaactggtatggttaccgaagaagatttcttcaacaaggttgatcaagttgccattatggccttcgatgatcaatgtacaggtgctaatccaagataccctttggtttctgaattgaagcaattgatgattgatgcctggaatggtgttgttccaaagttg

SEQ ID NO: 4 - DNA, 2049 bp, Saccharomyces cerevisiae
atgacaatcaaggaacataaagtagtttatgaagctcacaacgtaaaggctcttaaggctcctcaacatttttacaacagccaacccggcaagggttacgttactgatatgcaacattatcaagaaatgtatcaacaatctatcaatgagccagaaaaattctttgataagatggctaaggaatacttgcattgggatgctccatacaccaaagttcaatctggttcattgaacaatggtgatgttgcatggtttttgaacggtaaattgaatgcatcatacaattgtgttgacagacatgcctttgctaatcccgacaagccagctttgatctatgaagctgatgacgaatccgacaacaaaatcatcacatttggtgaattactcagaaaagtttcccaaatcgctggtgtcttaaaaagctggggcgttaagaaaggtgacacagtggctatctatttgccaatgattccagaagcggtcattgctatgttggctgtggctcgtattggtgctattcactctgttgtctttgctgggttctccgctggttcgttgaaagatcgtgtcgttgacgctaattctaaagtggtcatcacttgtgatgaaggtaaaagaggtggtaagaccatcaacactaaaaaaattgttgacgaaggtttgaacggagtcgatttggtttcccgtatcttggttttccaaagaactggtactgaaggtattccaatgaaggccggtagagattactggtggcatgaggaggccgctaagcagagaacttacctacctcctgtttcatgtgacgctgaagatcctctatttttattatacacttccggttccactggttctccaaagggtgtcgttcacactacaggtggttatttattaggtgccgctttaacaactagatacgtttttgatattcacccagaagatgttctcttcactgccggtgacgtcggctggatcacgggtcacacctatgctctatatggtccattaaccttgggtaccgcctcaataattttcgaatccactcctgcctacccagattatggtagatattggagaattatccaacgtcacaaggctacccatttctatgtggctccaactgctttaagattaatcaaacgtgtaggtgaagccgaaattgccaaatatgacacttcctcattacgtgtcttgggttccgtcggtgaaccaatctctccagacttatgggaatggtatcatgaaaaagtgggtaacaaaaactgtgtcatttgtgacactatgtggcaaacagagtctggttctcatttaattgctcctttggcaggtgctgtcccaacaaaacctggttctgctaccgtgccattctttggtattaacgcttgtatcattgaccctgttacaggtgtggaattagaaggtaatgatgtcgaaggtgtccttgccgttaaatcaccatggccatcaatggctagatctgtttggaaccaccacgaccgttacatggatacttacttgaaaccttatcctggtcactatttcacaggtgatggtgctggtagagatcatgatggttactactggatcaggggtagagttgacgacgttgtaaatgtttccggtcatagattatccacatcagaaattgaagcatctatctcaaatcacgaaaacgtctcggaagctgctgttgtcggtattccagatgaattgaccggtcaaaccgtcgttgcatatgtttccctaaaagatggttatctacaaaacaacgctactgaaggtgatgcagaacacatcacaccagataatttacgtagagaattgatcttacaagttaggggtgagattggtcctttcgcctcaccaaaaaccattattctagttagagatctaccaagaacaaggtcaggaaagattatgagaagagttctaagaaaggttgcttctaacgaagccgaacagctaggtgacctaactactttggccaacccagaagttgtacctgccatcatttctgctgtagagaaccaatttttctctcaaaaaaagaaa

SEQ ID NO: 5 - DNA, 20 bp, Artificial Sequence
  Amplification primer
attaaccctcactaaaggga

SEQ ID NO: 6 - DNA, 20 bp, Artificial Sequence
  Amplification primer
taatacgactcactataggg

SEQ ID NO: 7 - DNA, 20 bp, Artificial Sequence
  Amplification primer
cgaataaagtcgcggaaaaa

SEQ ID NO: 8 - DNA, 20 bp, Artificial Sequence
  Amplification primer
tcaagcgtctgacattgctg

SEQ ID NO: 9 - DNA, 20 bp, Artificial Sequence
  Amplification primer
tgagtttcttcatgggcgta

SEQ ID NO: 10 - DNA, 20 bp, Artificial Sequence
  Amplification primer
aaccacacgcacacgactct

SEQ ID NO: 11 - DNA, 27 bp, Artificial Sequence
  Amplification primer
ttttgttttctcttaactttgtatcct

SEQ ID NO: 12 - DNA, 19 bp, Artificial Sequence
  Amplification primer
ggatggggttcaccctctg

SEQ ID NO: 13 - DNA, 20 bp, Artificial Sequence
  Amplification primer
actcgccgatagtggaaacc

SEQ ID NO: 14 - DNA, 8457 bp, Artificial Sequence
  Expression construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgagcgccagatctgtttagcttgccttgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaagcatgggtactactcttgacgacacggcttaccgttaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgagttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcacataaatccggacgacagagctgcagaaaaatgggatggtcacatccacaattcgaaaaacatatgaaaaatgtccggtttacaaatgttccaaaacttgtccttgtacggttccttggctgaaattgatacttccgaaaagttgaacgaagctatggataagttgactgctgctcaagaacaattcagagaatacaatcaagaacaagtcgacaagattttcaaggctgttgctttggctgcttctcaaaacagagttgcttttgctaaatacgcccatgaagaaactcaaaagggtgttttcgaagataaggtcattaagaacgaattcgctgccgattacatctaccataagtactgtaatgataagaccgccggtatcatcgaatacgatgaagctaatggtttgatggaaattgctgaaccagttggtccagttgttggtattgctccagttactaatccaacctctaccatcatctacaagtccttgattgctttgaaaaccagaaactgcatcatcttctcaccacatccaggtgctcataaggcttcagtttttgttgttaaggtcttgcatcaagctgctgttaaggctggtgctccagaaaactgtattcaaattatcttcccaaagatggacttgaccaccgaattattgcatcatcaaaagaccagattcatttgggctactggtggtccaggtttggttcatgcttcttatacttcaggtaaaccagctttgggtggtggtcctggtaatgctcctgctttgattgacgaaacttgtgatatgaatgaagccgttggttccatcgttgtttctaagacttttgattgcggtatgatttgcgctactgaaaacgccgttgttgttgtagaatctgtctacgaaaatttcgttgccactatgaagaaaagaggtgcttactttatgaccccagaagaaacaaagaaggcctctaatttgttgttcggtgaaggtatgagattgaacgctaaagctgttggtcaaactgctaaaactttagctgaaatggctggtttcgaagtcccagaaaacactgttgttttgtgtggtgaagcttccgaagttaagtttgaagaacctatggctcacgaaaaattgaccactattttgggtatctacaaggccaaggatttcgatgatggtgttagattgtgcaaagaattggttacttttggtggtaagggtcatactgctgtcttgtacactaatcaaaacaacaaggacagaatcgaaaagtaccaaaacgaagttccagccttccatatcttggttgatatgccatcttctttgggttgcattggtgatatgtacaactttagattggctccagctttgactattacctgtggtactatgggtggtggttcttcttctgataatatcggtccaaaacacttgttgaacatcaagagagttggtatgagaagagaaaacatgttatggttcaagatcccaaagtccgtctacttcaaaagagccattttgtctgaagctttgtccgatttgagagatactcataagagagccattatcatcaccgatagaactatgactatgttgggtcaaaccgacaagatcattaaggcttgtgaaggtcatggtatggtctgtactgtttacgataaggttgttccagatccaaccattaagtgcattatggaaggtgtcaacgaaatgaacgttttcaaaccagatttggctattgcattaggtggtggtagtgctatggatgctgctaaaatgatgagattattctacgaatacccagaccaagacttgcaagatattgctactagattcgtcgacatcagaaaaagagttgtcggttgtccaaaattgggtagattgattaagaccttggtctgtattccaactacttctggtactggtgctgaagttactccttttgctgttgttacttctgaagaaggtagaaagtacccattggttgattacgaattgactccagatatggcaatcgttgatccagaatttgccgttggtatgccaaaaagattgacttcttggactggtattgatgctttgacccatgctattgaatcctacgtttctattatggctaccgatttcaccagaccttattctttgagagcagttggtttgatcttcgaatctttgtctttggcctacaacaatggtaaggatatcgaagccagagaaaagatgcataacgcttcagctattgctggtatggcatttgctaatgcttttttgggttgttgccattccgttgctcatcaattgggttctgtttaccatattccacatggtttggcaaacgctttgatgttgtctcacattattaagtacaacgccactgattccccagttaagatgggtacatttccacaatacaaatacccacaagccatgagacattacgcagaaattgcagaattgttgttgcctccaactcaagttgttaagatgaccgatgttgataaggtccaatacttgatcgacagagtcgaacaattgaaagctgatgtcggtattccaaagtccatcaaagaaactggtatggttaccgaagaagatttcttcaacaaggttgatcaagttgccattatggccttcgatgatcaatgtacaggtgctaatccaagataccctttggtttctgaattgaagcaattgatgattgatgcctggaatggtgttgttccaaagttggggggaggcgggggtggatggtctcacccacaattcgaaaagtgaggatccgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagcttcgcagtttacactctcatcgtcgctctcatcatcgcttccgttgttgttttccttagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgt

SEQ ID NO: 15 - DNA, 10102 bp, Artificial Sequence
  Expression construct
gaattcttattacgatcctcgcgccccctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataacgcgttctctagtgaaaaaccttgttggcataaaaaggctaattgattttcgagagtttcatactgtttttctgtaggccgtgtatctgaatgtacttttgctccattgcgatgacttagtaaagcacatctaaaacttttagcgttattgcgtaaaaaatcttgccagctttccccttttaaagggcaaaagtgagtatggtgcctatctaacatctcaatggctaaggcgtcgagcaaagcccgcttattttttacatgccaatacagtgtaggctgctctacaccaagcttctgggcgagtttacgggttgttaaaccttcgattccgacctcattaagcagctctaatgcgctgttaatcactttacttttatctaatctagacatatgaattaattcgggccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttagcgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacatggcggtaatgttggacatgagccaatataaatgtacatattatgatatggatacaacgtatgcaatgggccaagctcctcgagtaattcgcgccacttctaaataagcgaatttcttatgatttatgatttttattattaaataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcagatcaattcctcgatccctatcagtgatagagagtcgacaaagtcgagtttctcgatccctatcagtgatagagagtcgacaaagtcgagtttctcgatcgagaccactgcatgcatgtgctctgtatgtatataaaactcttgttttcttcttttctctaaatattctttccttatacattaggtcctttgtagcataaattactatacttctatagacacgcaaacacaaatacacacactaaattaccggatcaattcgggggatccaaaaatgaatcataaggttcatcatcaccatcaccacgcggccgccatgacaatcaaggaacataaagtagtttatgaagctcacaacgtaaaggctcttaaggctcctcaacatttttacaacagccaacccggcaagggttacgttactgatatgcaacattatcaagaaatgtatcaacaatctatcaatgagccagaaaaattctttgataagatggctaaggaatacttgcattgggatgctccatacaccaaagttcaatctggttcattgaacaatggtgatgttgcatggtttttgaacggtaaattgaatgcatcatacaattgtgttgacagacatgcctttgctaatcccgacaagccagctttgatctatgaagctgatgacgaatccgacaacaaaatcatcacatttggtgaattactcagaaaagtttcccaaatcgctggtgtcttaaaaagctggggcgttaagaaaggtgacacagtggctatctatttgccaatgattccagaagcggtcattgctatgttggctgtggctcgtattggtgctattcactctgttgtctttgctgggttctccgctggttcgttgaaagatcgtgtcgttgacgctaattctaaagtggtcatcacttgtgatgaaggtaaaagaggtggtaagaccatcaacactaaaaaaattgttgacgaaggtttgaacggagtcgatttggtttcccgtatcttggttttccaaagaactggtactgaaggtattccaatgaaggccggtagagattactggtggcatgaggaggccgctaagcagagaacttacctacctcctgtttcatgtgacgctgaagatcctctatttttattatacacttccggttccactggttctccaaagggtgtcgttcacactacaggtggttatttattaggtgccgctttaacaactagatacgtttttgatattcacccagaagatgttctcttcactgccggtgacgtcggctggatcacgggtcacacctatgctctatatggtccattaaccttgggtaccgcctcaataattttcgaatccactcctgcctacccagattatggtagatattggagaattatccaacgtcacaaggctacccatttctatgtggctccaactgctttaagattaatcaaacgtgtaggtgaagccgaaattgccaaatatgacacttcctcattacgtgtcttgggttccgtcggtgaaccaatctctccagacttatgggaatggtatcatgaaaaagtgggtaacaaaaactgtgtcatttgtgacactatgtggcaaacagagtctggttctcatttaattgctcctttggcaggtgctgtcccaacaaaacctggttctgctaccgtgccattctttggtattaacgcttgtatcattgaccctgttacaggtgtggaattagaaggtaatgatgtcgaaggtgtccttgccgttaaatcaccatggccatcaatggctagatctgtttggaaccaccacgaccgttacatggatacttacttgaaaccttatcctggtcactatttcacaggtgatggtgctggtagagatcatgatggttactactggatcaggggtagagttgacgacgttgtaaatgtttccggtcatagattatccacatcagaaattgaagcatctatctcaaatcacgaaaacgtctcggaagctgctgttgtcggtattccagatgaattgaccggtcaaaccgtcgttgcatatgtttccctaaaagatggttatctacaaaacaacgctactgaaggtgatgcagaacacatcacaccagataatttacgtagagaattgatcttacaagttaggggtgagattggtcctttcgcctcaccaaaaaccattattctagttagagatctaccaagaacaaggtcaggaaagattatgagaagagttctaagaaaggttgcttctaacgaagccgaacagctaggtgacctaactactttggccaacccagaagttgtacctgccatcatttctgctgtagagaaccaatttttctctcaaaaaaagaaatgatcactgcaggagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattgcgccagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaatttgcaagccttttgaaaagcaagcataaaagatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccggcagcttagtatataaatacacatgtacatacctctctccgtatcctcgtaatcattttcttgtatttatcgtcttttcgctgtaaaaactttatcacacttatctcaaatacacttattaaccgcttttactattatcttctacgctgacagtaatatcaaacagtgacacatattaaacacagtggtttctttgcataaacaccatcagcctcaagtcgtcaagtaaagatttcgtgttcatgcagatagataacaatctatatgttgataattagcgttgcctcatcaatgcgagatccgtttaaccggaccctagtgcacttaccccacgttcggtccactgtgtgccgaacatgctccttcactattttaacatgtggaattaattctcatgtttgacagcttatcatcgaactctaagaggtgatacttatttactgtaaaactgtgacgataaaaccggaaggaagaataagaaaactcgaactgatctataatgcctattttctgtaaagagtttaagctatgaaagcctcggcattttggccgctcctaggtagtgctttttttccaaggacaaaacagtttctttttcttgagcaggttttatgtttcggtaatcataaacaataaataaattatttcatttatgtttaaaaataaaaaataaaaaagtattttaaatttttaaaaaagttgattataagcatgtgaccttttgcaagcaattaaattttgcaatttgtgattttaggcaaaagttacaatttctggctcgtgtaatatatgtatgctaaagtgaacttttacaaagtcgatatggacttagtcaaaagaaattttcttaaaaatatatagcactagccaatttagcacttctttatgagatatattatagactttattaagccagatttgtgtattatatgtatttacccggcgaatcatggacatacattctgaaataggtaatattctctatggtgagacagcatagataacctaggatacaagttaaaagctagtactgttttgcagtaatttttttcttttttataagaatgttaccacctaaataagttataaagtcaatagttaagtttgatatttgattgtaaaataccgtaatatatttgcatgatcaaaaggctcaatgttgactagccagcatgtcaaccactatattgatcaccgatatatggacttccacaccaactagtaatatgacaataaattcaagatattcttcatgagaatggcccagcgatatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagt

SEQ ID NO: 16 - DNA, 5002 bp, Artificial Sequence
  Expression construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgagcgccagatctgtttagcttgccttgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaagcatgggtactactcttgacgacacggcttaccgttaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 17 - DNA, 8043 bp, Artificial Sequence
  Expression construct
gaattcttattacgatcctcgcgccccctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataacgcgttctctagtgaaaaaccttgttggcataaaaaggctaattgattttcgagagtttcatactgtttttctgtaggccgtgtatctgaatgtacttttgctccattgcgatgacttagtaaagcacatctaaaacttttagcgttattgcgtaaaaaatcttgccagctttccccttttaaagggcaaaagtgagtatggtgcctatctaacatctcaatggctaaggcgtcgagcaaagcccgcttattttttacatgccaatacagtgtaggctgctctacaccaagcttctgggcgagtttacgggttgttaaaccttcgattccgacctcattaagcagctctaatgcgctgttaatcactttacttttatctaatctagacatatgaattaattcgggccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttagcgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacatggcggtaatgttggacatgagccaatataaatgtacatattatgatatggatacaacgtatgcaatgggccaagctcctcgagtaattcgcgccacttctaaataagcgaatttcttatgatttatgatttttattattaaataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcagatcaattcctcgatccctatcagtgatagagagtcgacaaagtcgagtttctcgatccctatcagtgatagagagtcgacaaagtcgagtttctcgatcgagaccactgcatgcatgtgctctgtatgtatataaaactcttgttttcttcttttctctaaatattctttccttatacattaggtcctttgtagcataaattactatacttctatagacacgcaaacacaaatacacacactaaattaccggatcaattcgggggatccgtttaaacaggcctgttaacatcgatagcggccgctagggccctgcaggagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattgcgccagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaatttgcaagccttttgaaaagcaagcataaaagatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccggcagcttagtatataaatacacatgtacatacctctctccgtatcctcgtaatcattttcttgtatttatcgtcttttcgctgtaaaaactttatcacacttatctcaaatacacttattaaccgcttttactattatcttctacgctgacagtaatatcaaacagtgacacatattaaacacagtggtttctttgcataaacaccatcagcctcaagtcgtcaagtaaagatttcgtgttcatgcagatagataacaatctatatgttgataattagcgttgcctcatcaatgcgagatccgtttaaccggaccctagtgcacttaccccacgttcggtccactgtgtgccgaacatgctccttcactattttaacatgtggaattaattctcatgtttgacagcttatcatcgaactctaagaggtgatacttatttactgtaaaactgtgacgataaaaccggaaggaagaataagaaaactcgaactgatctataatgcctattttctgtaaagagtttaagctatgaaagcctcggcattttggccgctcctaggtagtgctttttttccaaggacaaaacagtttctttttcttgagcaggttttatgtttcggtaatcataaacaataaataaattatttcatttatgtttaaaaataaaaaataaaaaagtattttaaatttttaaaaaagttgattataagcatgtgaccttttgcaagcaattaaattttgcaatttgtgattttaggcaaaagttacaatttctggctcgtgtaatatatgtatgctaaagtgaacttttacaaagtcgatatggacttagtcaaaagaaattttcttaaaaatatatagcactagccaatttagcacttctttatgagatatattatagactttattaagccagatttgtgtattatatgtatttacccggcgaatcatggacatacattctgaaataggtaatattctctatggtgagacagcatagataacctaggatacaagttaaaagctagtactgttttgcagtaatttttttcttttttataagaatgttaccacctaaataagttataaagtcaatagttaagtttgatatttgattgtaaaataccgtaatatatttgcatgatcaaaaggctcaatgttgactagccagcatgtcaaccactatattgatcaccgatatatggacttccacaccaactagtaatatgacaataaattcaagatattcttcatgagaatggcccagcgatatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagt

SEQ ID NO: 18 - DNA, 8522 bp, Artificial Sequence
  Expression construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgagcgccagatctgtttagcttgccttgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaagcatgggtactactcttgacgacacggcttaccgttaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgagtactttcgtgtgtaggtattcttttagacgtttggttattgggtagatatgagagagagagagtgggtgggggaggagttggttgtaggagggacccctgggaggaagtgtagttgagttttccctgacgaatgaaaatacgtttttgagaagataatacaggaaaggtgtgtcggtgaatttccatctatccgaggatatgagtggaggagagtcgtgtgcgtgtggttaatttaggatcagtggaacacacaaagtaactaagacagagagacagagagaaaaatctggggaagagacaaagagtcagagtgtgtgagttattctgtattgtgaaatttttttgcccaactacataatattgctgaaactaattttacttaaaaagaaaagccaacaacgtccccagtaaaacttttctataaatatcagcagttttccctttcctccattcctcttcttgtcttttttcttactttcccttttttataccttttcattatcatcctttataattgtctaaccaacaactatatatctatcaaccatatgaaaaatgtccggtttacaaatgttccaaaacttgtccttgtacggttccttggctgaaattgatacttccgaaaagttgaacgaagctatggataagttgactgctgctcaagaacaattcagagaatacaatcaagaacaagtcgacaagattttcaaggctgttgctttggctgcttctcaaaacagagttgcttttgctaaatacgcccatgaagaaactcaaaagggtgttttcgaagataaggtcattaagaacgaattcgctgccgattacatctaccataagtactgtaatgataagaccgccggtatcatcgaatacgatgaagctaatggtttgatggaaattgctgaaccagttggtccagttgttggtattgctccagttactaatccaacctctaccatcatctacaagtccttgattgctttgaaaaccagaaactgcatcatcttctcaccacatccaggtgctcataaggcttcagtttttgttgttaaggtcttgcatcaagctgctgttaaggctggtgctccagaaaactgtattcaaattatcttcccaaagatggacttgaccaccgaattattgcatcatcaaaagaccagattcatttgggctactggtggtccaggtttggttcatgcttcttatacttcaggtaaaccagctttgggtggtggtcctggtaatgctcctgctttgattgacgaaacttgtgatatgaatgaagccgttggttccatcgttgtttctaagacttttgattgcggtatgatttgcgctactgaaaacgccgttgttgttgtagaatctgtctacgaaaatttcgttgccactatgaagaaaagaggtgcttactttatgaccccagaagaaacaaagaaggcctctaatttgttgttcggtgaaggtatgagattgaacgctaaagctgttggtcaaactgctaaaactttagctgaaatggctggtttcgaagtcccagaaaacactgttgttttgtgtggtgaagcttccgaagttaagtttgaagaacctatggctcacgaaaaattgaccactattttgggtatctacaaggccaaggatttcgatgatggtgttagattgtgcaaagaattggttacttttggtggtaagggtcatactgctgtcttgtacactaatcaaaacaacaaggacagaatcgaaaagtaccaaaacgaagttccagccttccatatcttggttgatatgccatcttctttgggttgcattggtgatatgtacaactttagattggctccagctttgactattacctgtggtactatgggtggtggttcttcttctgataatatcggtccaaaacacttgttgaacatcaagagagttggtatgagaagagaaaacatgttatggttcaagatcccaaagtccgtctacttcaaaagagccattttgtctgaagctttgtccgatttgagagatactcataagagagccattatcatcaccgatagaactatgactatgttgggtcaaaccgacaagatcattaaggcttgtgaaggtcatggtatggtctgtactgtttacgataaggttgttccagatccaaccattaagtgcattatggaaggtgtcaacgaaatgaacgttttcaaaccagatttggctattgcattaggtggtggtagtgctatggatgctgctaaaatgatgagattattctacgaatacccagaccaagacttgcaagatattgctactagattcgtcgacatcagaaaaagagttgtcggttgtccaaaattgggtagattgattaagaccttggtctgtattccaactacttctggtactggtgctgaagttactccttttgctgttgttacttctgaagaaggtagaaagtacccattggttgattacgaattgactccagatatggcaatcgttgatccagaatttgccgttggtatgccaaaaagattgacttcttggactggtattgatgctttgacccatgctattgaatcctacgtttctattatggctaccgatttcaccagaccttattctttgagagcagttggtttgatcttcgaatctttgtctttggcctacaacaatggtaaggatatcgaagccagagaaaagatgcataacgcttcagctattgctggtatggcatttgctaatgcttttttgggttgttgccattccgttgctcatcaattgggttctgtttaccatattccacatggtttggcaaacgctttgatgttgtctcacattattaagtacaacgccactgattccccagttaagatgggtacatttccacaatacaaatacccacaagccatgagacattacgcagaaattgcagaattgttgttgcctccaactcaagttgttaagatgaccgatgttgataaggtccaatacttgatcgacagagtcgaacaattgaaagctgatgtcggtattccaaagtccatcaaagaaactggtatggttaccgaagaagatttcttcaacaaggttgatcaagttgccattatggccttcgatgatcaatgtacaggtgctaatccaagataccctttggtttctgaattgaagcaattgatgattgatgcctggaatggtgttgttccaaagttggggggtggatggtctcacccacaattcgaaaagtgaggatccgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagcttcgcagtttacactctcatcgtcgctctcatcatcgcttccgttgttgttttccttagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 19 - DNA, 7922 bp, Artificial Sequence
  Expression construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgagcgccagatctgtttagcttgccttgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaagcatgggtactactcttgacgacacggcttaccgttaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgagtactttcgtgtgtaggtattcttttagacgtttggttattgggtagatatgagagagagagagtgggtgggggaggagttggttgtaggagggacccctgggaggaagtgtagttgagttttccctgacgaatgaaaatacgtttttgagaagataatacaggaaaggtgtgtcggtgaatttccatctatccgaggatatgagtggaggagagtcgtgtgcgtgtggttaatttaggatcagtggaacacacaaagtaactaagacagagagacagagagaaaaatctggggaagagacaaagagtcagagtgtgtgagttattctgtattgtgaaatttttttgcccaactacataatattgctgaaactaattttacttaaaaagaaaagccaacaacgtccccagtaaaacttttctataaatatcagcagttttccctttcctccattcctcttcttgtcttttttcttactttcccttttttataccttttcattatcatcctttataattgtctaaccaacaactatatatctatcaaccatatgaaaaatgggttggagccacccgcagttcgaaaaaggcggaggtatgacaatcaaggaacataaagtagtttatgaagctcacaacgtaaaggctcttaaggctcctcaacatttttacaacagccaacccggcaagggttacgttactgatatgcaacattatcaagaaatgtatcaacaatctatcaatgagccagaaaaattctttgataagatggctaaggaatacttgcattgggatgctccatacaccaaagttcaatctggttcattgaacaatggtgatgttgcatggtttttgaacggtaaattgaatgcatcatacaattgtgttgacagacatgcctttgctaatcccgacaagccagctttgatctatgaagctgatgacgaatccgacaacaaaatcatcacatttggtgaattactcagaaaagtttcccaaatcgctggtgtcttaaaaagctggggcgttaagaaaggtgacacagtggctatctatttgccaatgattccagaagcggtcattgctatgttggctgtggctcgtattggtgctattcactctgttgtctttgctgggttctccgctggttcgttgaaagatcgtgtcgttgacgctaattctaaagtggtcatcacttgtgatgaaggtaaaagaggtggtaagaccatcaacactaaaaaaattgttgacgaaggtttgaacggagtcgatttggtttcccgtatcttggttttccaaagaactggtactgaaggtattccaatgaaggccggtagagattactggtggcatgaggaggccgctaagcagagaacttacctacctcctgtttcatgtgacgctgaagatcctctatttttattatacacttccggttccactggttctccaaagggtgtcgttcacactacaggtggttatttattaggtgccgctttaacaactagatacgtttttgatattcacccagaagatgttctcttcactgccggtgacgtcggctggatcacgggtcacacctatgctctatatggtccattaaccttgggtaccgcctcaataattttcgaatccactcctgcctacccagattatggtagatattggagaattatccaacgtcacaaggctacccatttctatgtggctccaactgctttaagattaatcaaacgtgtaggtgaagccgaaattgccaaatatgacacttcctcattacgtgtcttgggttccgtcggtgaaccaatctctccagacttatgggaatggtatcatgaaaaagtgggtaacaaaaactgtgtcatttgtgacactatgtggcaaacagagtctggttctcatttaattgctcctttggcaggtgctgtcccaacaaaacctggttctgctaccgtgccattctttggtattaacgcttgtatcattgaccctgttacaggtgtggaattagaaggtaatgatgtcgaaggtgtccttgccgttaaatcaccatggccatcaatggctagatctgtttggaaccaccacgaccgttacatggatacttacttgaaaccttatcctggtcactatttcacaggtgatggtgctggtagagatcatgatggttactactggatcaggggtagagttgacgacgttgtaaatgtttccggtcatagattatccacatcagaaattgaagcatctatctcaaatcacgaaaacgtctcggaagctgctgttgtcggtattccagatgaattgaccggtcaaaccgtcgttgcatatgtttccctaaaagatggttatctacaaaacaacgctactgaaggtgatgcagaacacatcacaccagataatttacgtagagaattgatcttacaagttaggggtgagattggtcctttcgcctcaccaaaaaccattattctagttagagatctaccaagaacaaggtcaggaaagattatgagaagagttctaagaaaggttgcttctaacgaagccgaacagctaggtgacctaactactttggccaacccagaagttgtacctgccatcatttctgctgtagagaaccaatttttctctcaaaaaaagaaatgaggatccgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagcttcgcagtttacactctcatcgtcgctctcatcatcgcttccgttgttgttttccttagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 20 - DNA, 5463 bp, Artificial Sequence
  Expression construct
ggccgcgtattcaattcctattctaaatggcttttatttctattacaactattagctctaaatccatatcctcataagcagcaatcaattctatctatactttaaaatgctcgagtactttcgtgtgtaggtattcttttagacgtttggttattgggtagatatgagagagagagagtgggtgggggaggagttggttgtaggagggacccctgggaggaagtgtagttgagttttccctgacgaatgaaaatacgtttttgagaagataatacaggaaaggtgtgtcggtgaatttccatctatccgaggatatgagtggaggagagtcgtgtgcgtgtggttaatttaggatcagtggaacacacaaagtaactaagacagagagacagagagaaaaatctggggaagagacaaagagtcagagtgtgtgagttattctgtattgtgaaatttttttgcccaactacataatattgctgaaactaattttacttaaaaagaaaagccaacaacgtccccagtaaaacttttctataaatatcagcagttttccctttcctccattcctcttcttgtcttttttcttactttcccttttttataccttttcattatcatcctttataattgtctaaccaacaactatatatctatcaaccatatgaaaaatgtccggtttacaaatgttccaaaacttgtccttgtacggttccttggctgaaattgatacttccgaaaagttgaacgaagctatggataagttgactgctgctcaagaacaattcagagaatacaatcaagaacaagtcgacaagattttcaaggctgttgctttggctgcttctcaaaacagagttgcttttgctaaatacgcccatgaagaaactcaaaagggtgttttcgaagataaggtcattaagaacgaattcgctgccgattacatctaccataagtactgtaatgataagaccgccggtatcatcgaatacgatgaagctaatggtttgatggaaattgctgaaccagttggtccagttgttggtattgctccagttactaatccaacctctaccatcatctacaagtccttgattgctttgaaaaccagaaactgcatcatcttctcaccacatccaggtgctcataaggcttcagtttttgttgttaaggtcttgcatcaagctgctgttaaggctggtgctccagaaaactgtattcaaattatcttcccaaagatggacttgaccaccgaattattgcatcatcaaaagaccagattcatttgggctactggtggtccaggtttggttcatgcttcttatacttcaggtaaaccagctttgggtggtggtcctggtaatgctcctgctttgattgacgaaacttgtgatatgaatgaagccgttggttccatcgttgtttctaagacttttgattgcggtatgatttgcgctactgaaaacgccgttgttgttgtagaatctgtctacgaaaatttcgttgccactatgaagaaaagaggtgcttactttatgaccccagaagaaacaaagaaggcctctaatttgttgttcggtgaaggtatgagattgaacgctaaagctgttggtcaaactgctaaaactttagctgaaatggctggtttcgaagtcccagaaaacactgttgttttgtgtggtgaagcttccgaagttaagtttgaagaacctatggctcacgaaaaattgaccactattttgggtatctacaaggccaaggatttcgatgatggtgttagattgtgcaaagaattggttacttttggtggtaagggtcatactgctgtcttgtacactaatcaaaacaacaaggacagaatcgaaaagtaccaaaacgaagttccagccttccatatcttggttgatatgccatcttctttgggttgcattggtgatatgtacaactttagattggctccagctttgactattacctgtggtactatgggtggtggttcttcttctgataatatcggtccaaaacacttgttgaacatcaagagagttggtatgagaagagaaaacatgttatggttcaagatcccaaagtccgtctacttcaaaagagccattttgtctgaagctttgtccgatttgagagatactcataagagagccattatcatcaccgatagaactatgactatgttgggtcaaaccgacaagatcattaaggcttgtgaaggtcatggtatggtctgtactgtttacgataaggttgttccagatccaaccattaagtgcattatggaaggtgtcaacgaaatgaacgttttcaaaccagatttggctattgcattaggtggtggtagtgctatggatgctgctaaaatgatgagattattctacgaatacccagaccaagacttgcaagatattgctactagattcgtcgacatcagaaaaagagttgtcggttgtccaaaattgggtagattgattaagaccttggtctgtattccaactacttctggtactggtgctgaagttactccttttgctgttgttacttctgaagaaggtagaaagtacccattggttgattacgaattgactccagatatggcaatcgttgatccagaatttgccgttggtatgccaaaaagattgacttcttggactggtattgatgctttgacccatgctattgaatcctacgtttctattatggctaccgatttcaccagaccttattctttgagagcagttggtttgatcttcgaatctttgtctttggcctacaacaatggtaaggatatcgaagccagagaaaagatgcataacgcttcagctattgctggtatggcatttgctaatgcttttttgggttgttgccattccgttgctcatcaattgggttctgtttaccatattccacatggtttggcaaacgctttgatgttgtctcacattattaagtacaacgccactgattccccagttaagatgggtacatttccacaatacaaatacccacaagccatgagacattacgcagaaattgcagaattgttgttgcctccaactcaagttgttaagatgaccgatgttgataaggtccaatacttgatcgacagagtcgaacaattgaaagctgatgtcggtattccaaagtccatcaaagaaactggtatggttaccgaagaagatttcttcaacaaggttgatcaagttgccattatggccttcgatgatcaatgtacaggtgctaatccaagataccctttggtttctgaattgaagcaattgatgattgatgcctggaatggtgttgttccaaagttggggggtggatggtctcacccacaattcgaaaagtgaggatccgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagcttcgcagtttacactctcatcgtcgctctcatcatcgcttccgttgttgttttccttagatctgtttagcttgccttgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaactatgggtaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgattttatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattcatcgataaaatgtgtatattagtttaaaaagttgtatgtaataaaagtaaaatttaatattttggatgaaaaaaaccatttttagactttttcttaactagaatgctga

SEQ ID NO: 21 - DNA, 4407 bp, Artificial Sequence
  Expression construct
ggccgcgtattcaattcctattctaaatggcttttatttctattacaactattagctctaaatccatatcctcataagcagcaatcaattctatctatactttaaaatgctcgagtactttcgtgtgtaggtattcttttagacgtttggttattgggtagatatgagagagagagagtgggtgggggaggagttggttgtaggagggacccctgggaggaagtgtagttgagttttccctgacgaatgaaaatacgtttttgagaagataatacaggaaaggtgtgtcggtgaatttccatctatccgaggatatgagtggaggagagtcgtgtgcgtgtggttaatttaggatcagtggaacacacaaagtaactaagacagagagacagagagaaaaatctggggaagagacaaagagtcagagtgtgtgagttattctgtattgtgaaatttttttgcccaactacataatattgctgaaactaattttacttaaaaagaaaagccaacaacgtccccagtaaaacttttctataaatatcagcagttttccctttcctccattcctcttcttgtcttttttcttactttcccttttttataccttttcattatcatcctttataattgtctaaccaacaactatatatctatcaaccatatgaaaaatgggttggagccacccgcagttcgaaaaaggcggaggtatgacaatcaaggaacataaagtagtttatgaagctcacaacgtaaaggctcttaaggctcctcaacatttttacaacagccaacccggcaagggttacgttactgatatgcaacattatcaagaaatgtatcaacaatctatcaatgagccagaaaaattctttgataagatggctaaggaatacttgcattgggatgctccatacaccaaagttcaatctggttcattgaacaatggtgatgttgcatggtttttgaacggtaaattgaatgcatcatacaattgtgttgacagacatgcctttgctaatcccgacaagccagctttgatctatgaagctgatgacgaatccgacaacaaaatcatcacatttggtgaattactcagaaaagtttcccaaatcgctggtgtcttaaaaagctggggcgttaagaaaggtgacacagtggctatctatttgccaatgattccagaagcggtcattgctatgttggctgtggctcgtattggtgctattcactctgttgtctttgctgggttctccgctggttcgttgaaagatcgtgtcgttgacgctaattctaaagtggtcatcacttgtgatgaaggtaaaagaggtggtaagaccatcaacactaaaaaaattgttgacgaaggtttgaacggagtcgatttggtttcccgtatcttggttttccaaagaactggtactgaaggtattccaatgaaggccggtagagattactggtggcatgaggaggccgctaagcagagaacttacctacctcctgtttcatgtgacgctgaagatcctctatttttattatacacttccggttccactggttctccaaagggtgtcgttcacactacaggtggttatttattaggtgccgctttaacaactagatacgtttttgatattcacccagaagatgttctcttcactgccggtgacgtcggctggatcacgggtcacacctatgctctatatggtccattaaccttgggtaccgcctcaataattttcgaatccactcctgcctacccagattatggtagatattggagaattatccaacgtcacaaggctacccatttctatgtggctccaactgctttaagattaatcaaacgtgtaggtgaagccgaaattgccaaatatgacacttcctcattacgtgtcttgggttccgtcggtgaaccaatctctccagacttatgggaatggtatcatgaaaaagtgggtaacaaaaactgtgtcatttgtgacactatgtggcaaacagagtctggttctcatttaattgctcctttggcaggtgctgtcccaacaaaacctggttctgctaccgtgccattctttggtattaacgcttgtatcattgaccctgttacaggtgtggaattagaaggtaatgatgtcgaaggtgtccttgccgttaaatcaccatggccatcaatggctagatctgtttggaaccaccacgaccgttacatggatacttacttgaaaccttatcctggtcactatttcacaggtgatggtgctggtagagatcatgatggttactactggatcaggggtagagttgacgacgttgtaaatgtttccggtcatagattatccacatcagaaattgaagcatctatctcaaatcacgaaaacgtctcggaagctgctgttgtcggtattccagatgaattgaccggtcaaaccgtcgttgcatatgtttccctaaaagatggttatctacaaaacaacgctactgaaggtgatgcagaacacatcacaccagataatttacgtagagaattgatcttacaagttaggggtgagattggtcctttcgcctcaccaaaaaccattattctagttagagatctaccaagaacaaggtcaggaaagattatgagaagagttctaagaaaggttgcttctaacgaagccgaacagctaggtgacctaactactttggccaacccagaagttgtacctgccatcatttctgctgtagagaaccaatttttctctcaaaaaaagaaatgaggatccgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagcttcgcagtttacactctcatcgtcgctctcatcatcgcttccgttgttgttttccttagatctgtttagcttgccttgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaagcatgggtactactcttgacgacacggcttaccgttaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattcatcgataaaatgtgtatattagtttaaaaagttgtatgtaataaaagtaaaatttaatattttggatgaaaaaaaccatttttagactttttcttaactagaatgctga

SEQ ID NO: 22 - DNA, 8653 bp, Artificial Sequence
  Expression construct
gaattcttattacgatcctcgcgccccctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataacgcgttctctagtgaaaaaccttgttggcataaaaaggctaattgattttcgagagtttcatactgtttttctgtaggccgtgtatctgaatgtacttttgctccattgcgatgacttagtaaagcacatctaaaacttttagcgttattgcgtaaaaaatcttgccagctttccccttttaaagggcaaaagtgagtatggtgcctatctaacatctcaatggctaaggcgtcgagcaaagcccgcttattttttacatgccaatacagtgtaggctgctctacaccaagcttctgggcgagtttacgggttgttaaaccttcgattccgacctcattaagcagctctaatgcgctgttaatcactttacttttatctaatctagacatatgaattaattcgggccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttagcgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacatggcggtaatgttggacatgagccaatataaatgtacatattatgatatggatacaacgtatgcaatgggccaagctcctcgagtaattcgcgccacttctaaataagcgaatttcttatgatttatgatttttattattaaataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcagatcaattcctcgatccctatcagtgatagagagtcgacaaagtcgagtttctcgatccctatcagtgatagagagtcgacaaagtcgagtttctcgatcgagaccactgcatgcatgtgctctgtatgtatataaaactcttgttttcttcttttctctaaatattctttccttatacattaggtcctttgtagcataaattactatacttctatagacacgcaaacacaaatacacacactaaattaccggatcaattcgggggatccaaaaatgaatcataaggttcatcatcaccatcaccacgcggccgccatggttgaacaagctaaattgtctgatccaaacgctgaatacaccatgagagatttgtctgctgaaaccattgatattaccaatccaagaggtggtgttagagatgctgaaattactgatgttcaaaccaccatggttgatggtaactacccatggattttggttagagtttacactgatgctggtgttgttggtactggtgaagcttattggggtggtggtgatactgctattattgaaagaatgaagccattcttggtcggtgaaaacccattggatattgacagattatacgaacacttggtccaaaagatgtctggtgaaggttctgtttctggtaaggttatttctgccatttccggtattgaaatcgccttgcatgatgttgctggtaagttgttggatgttccagcttatcaattggttggtggtaagtacagagatgaagttagagtctattgtgacttgcacactgaagatgaagctaatcctcaagcttgtgctgaagaaggtgtaagagttgttgaagaattgggttacgatgccatcaagttcgatttggatgtaccatctggtcacgaaaaggatagagctaatagacatttgagaaacccagaaatcgaccacaaggttgaaatagttgaagctgttactgaagccgttggtgatagagctgatgttgcatttgattgtcattggtcttttactggtggttccgctaaaagattggcttctgaattggaagattacgacgtttggtggttggaagatccagttccaccagaaaatcatgatgtccaaaagttggttactcaatctactactaccccaattgctgttggtgaaaacgtttacagaaagttcggtcaaagaaccttgttggaaccacaagccgttgatattattgctccagatttgccaagagtcggtggtatgagagaaactagaaaaattgctgatttggccgacatgtactacattccagttgctatgcataacgtcagttctccaattggtactatggcttctgctcaagttgctgctgctattccaaattctttggctttggaataccactcctaccaattaggttggtgggaagatttggttgaagaagatgatttgatccaaaacggtcacatggaaattccagaaaaaccaggtttgggtttgaccttggatttggacgctgttgaagctcacatggttgaaggtgaaactttgttcgatgaagaatgatcactgcaggagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgcactagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgagagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaagccttttgaaaagcaagcataaaagatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccggcagcttagtatataaatacacatgtacatacctctctccgtatcctcgtaatcattttcttgtatttatcgtcttttcgctgtaaaaactttatcacacttatctcaaatacacttattaaccgcttttactattatcttctacgctgacagtaatatcaaacagtgacacatattaaacacagtggtttctttgcataaacaccatcagcctcaagtcgtcaagtaaagatttcgtgttcatgcagatagataacaatctatatgttgataattagcgttgcctcatcaatgcgagatccgtttaaccggaccctagtgcacttaccccacgttcggtccactgtgtgccgaacatgctccttcactattttaacatgtggaattaattctcatgtttgacagcttatcatcgaactctaagaggtgatacttatttactgtaaaactgtgacgataaaaccggaaggaagaataagaaaactcgaactgatctataatgcctattttctgtaaagagtttaagctatgaaagcctcggcattttggccgctcctaggtagtgctttttttccaaggacaaaacagtttctttttcttgagcaggttttatgtttcggtaatcataaacaataaataaattatttcatttatgtttaaaaataaaaaataaaaaagtattttaaatttttaaaaaagttgattataagcatgtgaccttttgcaagcaattaaattttgcaatttgtgattttaggcaaaagttacaatttctggctcgtgtaatatatgtatgctaaagtgaacttttacaaagtcgatatggacttagtcaaaagaaattttcttaaaaatatatagcactagccaatttagcacttctttatgagatatattatagactttattaagccagatttgtgtattatatgtatttacccggcgaatcatggacatacattctgaaataggtaatattctctatggtgagacagcatagataacctaggatacaagttaaaagctagtactgttttgcagtaatttttttcttttttataagaatgttaccacctaaataagttataaagtcaatagttaagtttgatatttgattgtaaaataccgtaatatatttgcatgatcaaaaggctcaatgttgactagccagcatgtcaaccactatattgatcaccgatatatggacttccacaccaactagtaatatgacaataaattcaagatattcttcatgagaatggcccagcgatatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagt

SEQ ID NO: 23 - DNA, 8408 bp, Artificial Sequence
  Expression construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgagcgccagatctgtttagcttgccttgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaagcatgggtactactcttgacgacacggcttaccgttaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgagttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcacataaatccggacgacagagctgcagaaaaatgggatggtcacatccacaattcgaaaaacatatgtccggtttacaaatgttccaaaacttgtccttgtacggttccttggctgaaattgatacttccgaaaagttgaacgaagctatggataagttgactgctgctcaagaacaattcagagaatacaatcaagaacaagtcgacaagattttcaaggctgttgctttggctgcttctcaaaacagagttgcttttgctaaatacgcccatgaagaaactcaaaagggtgttttcgaagataaggtcattaagaacgaattcgctgccgattacatctaccataagtactgtaatgataagaccgccggtatcatcgaatacgatgaagctaatggtttgatggaaattgctgaaccagttggtccagttgttggtattgctccagttactaatccaacctctaccatcatctacaagtccttgattgctttgaaaaccagaaactgcatcatcttctcaccacatccaggtgctcataaggcttcagtttttgttgttaaggtcttgcatcaagctgctgttaaggctggtgctccagaaaactgtattcaaattatcttcccaaagatggacttgaccaccgaattattgcatcatcaaaagaccagattcatttgggctactggtggtccaggtttggttcatgcttcttatacttcaggtaaaccagctttgggtggtggtcctggtaatgctcctgctttgattgacgaaacttgtgatatgaatgaagccgttggttccatcgttgtttctaagacttttgattgcggtatgatttgcgctactgaaaacgccgttgttgttgtagaatctgtctacgaaaatttcgttgccactatgaagaaaagaggtgcttactttatgaccccagaagaaacaaagaaggcctctaatttgttgttcggtgaaggtatgagattgaacgctaaagctgttggtcaaactgctaaaactttagctgaaatggctggtttcgaagtcccagaaaacactgttgttttgtgtggtgaagcttccgaagttaagtttgaagaacctatggctcacgaaaaattgaccactattttgggtatctacaaggccaaggatttcgatgatggtgttagattgtgcaaagaattggttacttttggtggtaagggtcatactgctgtcttgtacactaatcaaaacaacaaggacagaatcgaaaagtaccaaaacgaagttccagccttccatatcttggttgatatgccatcttctttgggttgcattggtgatatgtacaactttagattggctccagctttgactattacctgtggtactatgggtggtggttcttcttctgataatatcggtccaaaacacttgttgaacatcaagagagttggtatgagaagagaaaacatgttatggttcaagatcccaaagtccgtctacttcaaaagagccattttgtctgaagctttgtccgatttgagagatactcataagagagccattatcatcaccgatagaactatgactatgttgggtcaaaccgacaagatcattaaggcttgtgaaggtcatggtatggtctgtactgtttacgataaggttgttccagatccaaccattaagtgcattatggaaggtgtcaacgaaatgaacgttttcaaaccagatttggctattgcattaggtggtggtagtgctatggatgctgctaaaatgatgagattattctacgaatacccagaccaagacttgcaagatattgctactagattcgtcgacatcagaaaaagagttgtcggttgtccaaaattgggtagattgattaagaccttggtctgtattccaactacttctggtactggtgctgaagttactccttttgctgttgttacttctgaagaaggtagaaagtacccattggttgattacgaattgactccagatatggcaatcgttgatccagaatttgccgttggtatgccaaaaagattgacttcttggactggtattgatgctttgacccatgctattgaatcctacgtttctattatggctaccgatttcaccagaccttattctttgagagcagttggtttgatcttcgaatctttgtctttggcctacaacaatggtaaggatatcgaagccagagaaaagatgcataacgcttcagctattgctggtatggcatttgctaatgcttttttgggttgttgccattccgttgctcatcaattgggttctgtttaccatattccacatggtttggcaaacgctttgatgttgtctcacattattaagtacaacgccactgattccccagttaagatgggtacatttccacaatacaaatacccacaagccatgagacattacgcagaaattgcagaattgttgttgcctccaactcaagttgttaagatgaccgatgttgataaggtccaatacttgatcgacagagtcgaacaattgaaagctgatgtcggtattccaaagtccatcaaagaaactggtatggttaccgaagaagatttcttcaacaaggttgatcaagttgccattatggccttcgatgatcaatgtacaggtgctaatccaagataccctttggtttctgaattgaagcaattgatgattgatgcctggaatggtgttgttccaaagttgtaggatccgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagcttcgcagtttacactctcatcgtcgctctcatcatcgcttccgttgttgttttccttagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 24 - DNA, 6624 bp, Artificial Sequence
  Expression construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgagcgccagatctgtttagcttgccttgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaagcatgggtactactcttgacgacacggcttaccgttaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgagttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcacataaatccggacgacagagctgcagaaaaatgggatggtcacatccacaattcgaaaaacatatgcactaccatcaattggccgtttctggtgaaagaagattgactgcttctagagattctactacctacgatttgacttctgctgatgctgatttgagaacttttggtgatttggctagagttgcttctattgctagaacctctgttgatagattggctgctgaattgactgaagatgctgatgttgttgatgatgccttcgttgatagacatgctactgttccagttgatgctgaagaaatttgggctgctggtgttacctatcaaatttctgaacaagccagagaagaagaatcctctatgccagatatgtacttcgatgtttacgatgctgatagaccagaagttttctttaaggctactccatctagaactgttgaaccaggtgatgctattggtgttagaggtgattctgaatgggatgttccagaaccagaattgggtatagttttgagaagaggtgaaatcgttggttacactgttggtaacgatgtctcctctagatcaattgaaggtgaaaacccattatacttgccacaagctaaggtttacgatagatgctgttctattggtccatgtgttgttactccagaagatgttgaagatccacacgaattggaaatgtccatgaccattgaaagagatggtgaagttatctacgatgatgctaccaacacttccgaaatggttagatcatgtgacgaattggtttcttacttcaccagacataataccgttccagaattagccgttattttgaccggtacttctttggttcctgaacaaccatttgacttgcaagaaggtgaccacgttgatattaccattgaaggtattggtactttgtccaactctgttactaccgtctgatcaggatccgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagcttcgcagtttacactctcatcgtcgctctcatcatcgcttccgttgttgttttccttagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 25 - DNA, 42 bp, Artificial Sequence
  Amplification primer
ccctgcagtgatcatttctttttttgagagaaaaattggttc

SEQ ID NO: 26 - DNA, 37 bp, Artificial Sequence
  Amplification primer
cacgcggccgccatgacaatcaaggaacataaagtag

SEQ ID NO: 27 - DNA, 75 bp, Artificial Sequence
  Amplification primer
gacattaacctataaaaataggcgtatcacgaggccctttcgtcttcaagaattgcgccagatctgtttagcttg

SEQ ID NO: 28 - DNA, 78 bp, Artificial Sequence
  Amplification primer
gttattttacagattttatgtttagatcttttatgcttgcttttcaaaaggcttgcaaattcgagctcgttttcgaca

SEQ ID NO: 29 - DNA, 20 bp, Artificial Sequence
  Amplification primer
ctgggtgagcaaaaacagga

SEQ ID NO: 30 - DNA, 20 bp, Artificial Sequence
  Amplification primer
gacaattcaacgcgtctgtg

SEQ ID NO: 31 - DNA, 72 bp, Artificial Sequence
  Amplification primer
ggcttaactatgcggcatcagagcagattgtactgagagtgcaccatatcgagcgccagatctgtttagctt

SEQ ID NO: 32 - DNA, 74 bp, Artificial Sequence
  Amplification primer
cgtttacaatttcctgatgcggtattttctccttacgcatctgtgcggtatttcaattcgagctcgttttcgac

SEQ ID NO: 33 - DNA, 20 bp, Artificial Sequence
  Amplification primer
cgttggagtccacgttcttt

SEQ ID NO: 34 - DNA, 45 bp, Artificial Sequence
  Amplification primer
ccggacgacagagcatatgaaaaatgtccggtttacaaatgttcc

SEQ ID NO: 35 - DNA, 77 bp, Artificial Sequence
  Amplification primer
tcggatcctcacttttcgaattgtgggtgagaccatccacccccgcctccccccaactttggaacaacaccattcca

SEQ ID NO: 36 - DNA, 57 bp, Artificial Sequence
  Amplification primer
acaaactgtacaatcaatcaatcaatcatcacataaatccggacgacagagcatatg

SEQ ID NO: 37 - DNA, 73 bp, Artificial Sequence
  Amplification primer
gcggatgtggggggagggcgtgaatgtaagcgtgacataactaattacatgactcggatcctcacttttcgaa

SEQ ID NO: 38 - DNA, 20 bp, Artificial Sequence
  Amplification primer
ctacaaggccaaggatttcg

SEQ ID NO: 39 - DNA, 20 bp, Artificial Sequence
  Amplification primer
gacggactttgggatcttga

SEQ ID NO: 40 - DNA, 20 bp, Artificial Sequence
  Amplification primer
gcagttttccctttcctcca

SEQ ID NO: 41 - DNA, 64 bp, Artificial Sequence
  Ampliciation primer
atgggttggagccacccgcagttcgaaaaaggcggaggtatgacaatcaaggaacataaagtag

SEQ ID NO: 42 - DNA, 92 bp, Artificial Sequence
  Amplification primer
gcggatgtggggggagggcgtgaatgtaagcgtgacataactaattacatgactcggatcctcatttctttttttgagagaaaaattggttc

SEQ ID NO: 43 - DNA, 74 bp, Artificial Sequence
  Amplification primer
catcctttataattgtctaaccaacaactatatatctatcaaccatatgaaaaatgggttggagccacccgcag

SEQ ID NO: 44 - DNA, 20 bp, Artificial Sequence
  Amplification primer
tactttggccaacccagaag

SEQ ID NO: 45 - DNA, 747 bp, Artificial Sequence
  Promoter construct
gcggccgcgccagggttttcccagtcacgacgttgtaaaacgacggccagtgagcgcgcgtaatacgactcactatagggcgaattgggtaccgggccccccctcgagtactttcgtgtgtaggtattcttttagacgtttggttattgggtagatatgagagagagagagtgggtgggggaggagttggttgtaggagggacccctgggaggaagtgtagttgagttttccctgacgaatgaaaatacgtttttgagaagataatacaggaaaggtgtgtcggtgaatttccatctatccgaggatatgagtggaggagagtcgtgtgcgtgtggttaatttaggatcagtggaacacacaaagtaactaagacagagagacagagagaaaaatctggggaagagacaaagagtcagagtgtgtgagttattctgtattgtgaaatttttttgcccaactacataatattgctgaaactaattttacttaaaaagaaaagccaacaacgtccccagtaaaacttttctataaatatcagcagttttccctttcctccattcctcttcttgtcttttttcttactttcccttttttataccttttcattatcatcctttataattgtctaaccaacaactatatatctatcaaccatatgaaaaatgtccggtttacaaatgttccaaaacttgtccttgtacggttccttggctgaaattgatacttccgaaaagttggcggccgc

(19) United States
(12) Patent Application Publication
(10) Pub. No.: US 2015/0079652 A1
(43) Pub. Date: Mar. 19, 2015

(54) NOVEL YEAST STRAINS

(71) Applicant: SHELL OIL COMPANY, Houston, TX (US)

(72) Inventors: Annette Victoria Angeles ALCASABAS, Cambridge (GB); Nadim ASHRAF, Katy, TX (US); George Robert LEE, Chester (GB); David Alexander PARKER, Chester (GB)

(21) Appl. No.: 14/486,377

(22) Filed: Sep. 15, 2014

(30) Foreign Application Priority Data
Sep. 19, 2013 (EP) 13185178.4

(51) Int. Cl. C12P 7/06; C12N 1/16

(52) U.S. Cl. CPC C12P 7/06; C12N 1/16
USPC 435/161; 435/254.21; 435/471

(57) ABSTRACT

Sacchromyces cerevisiae strains modified to express an adhE (alcohol/aldehyde dehydrogenase) enzyme having the amino acid sequence SEQ ID NO:1, or a functional variant thereof capable of catalysing the conversion of acetyl CoA to ethanol, and/or to overexpress an ACS2 (acetyl-CoA synthetase) enzyme having the amino acid sequence SEQ ID NO:2, or a functional variant thereof capable of catalysing the conversion of acetate to acetyl CoA. Such cells have an improved ability to grow on or in acetate-containing media compared to cells not according to the invention.

### RELATED APPLICATION

This application claims priority to European Application No. 13185178.4, filed on Sep. 19, 2013, the disclosure of which is incorporated herein by reference in its entirety.

### TECHNICAL FIELD

The present disclosure relates to novel yeast strains having improved resistance to acetate.

### BACKGROUND

Significant amounts of acetic acid are released upon hydrolysis of lignocellulosic biomass. For example, acetate is a byproduct of sugar fermentation and is present in significant amounts in hydrolyzed lignocellulosic biomass, the starting material for industrial ethanol production.

The undissociated forms of weak acids such as acetic acid can diffuse across the cell membrane of yeast cells and affect the intracellular pH (pHi) homeostasis. To control the influx of acids and maintain the pH, at a near-neutral value, excess protons have to be expelled by the cells, which require additional ATP synthesis via sugar fermentation. Eventually, the ATP production rate and the capacity of the ATPase become limiting, resulting in cessation of growth or even cell death.

It would be desirable to improve resistance of yeast cells to acetic acid in their growth media. Yeast cells are used industrially to carry out hydrolysis of lignocellulosic biomass.

Akamatsu et al. (J. Biosci. Bioeng. (2000) 90 555-560) have explored the role of various genes, including the acety-CoA synthetase isoform 2 (ACS2), in acetate production by sake yeast. Expression in other yeast strains was not investigated. A small increase (4% after 13 days) in alcohol production was observed. Boxma et al. (Mol. Microbiol. (2004) 51 1389-1399) described the identification of the bifunctional alcohol/aldehyde dehydrogenase gene in the hydrogenosome-containing yeast Piromyces sp. E2, the first time this gene was identified in a non-bacterial organism.

### SUMMARY

The present disclosure provides novel yeast strains expressing an adhE (bifunctional alcohol/aldehyde dehydrogenase) enzyme having the amino acid sequence SEQ ID NO: 1, or a functional variant thereof capable of catalysing the conversion of acetyl CoA to ethanol, and/or overexpressing an ACS2 (acetyl-CoA synthetase isoform 2) enzyme having the amino acid sequence SEQ ID NO:2, or a functional variant thereof capable of catalysing the conversion of acetate to acetyl CoA. Such cells have an improved ability to grow on or in acetate-containing media compared to cells not according to the invention. Furthermore, advantageously, cultures of such cells show an increase in ethanol production over about 24 hours compared to cells not according to the invention.

### DETAILED DESCRIPTION

Unless otherwise defined herein, scientific and technical terms used herein will have the meanings that are commonly understood by those of ordinary skill in the art.

Generally, nomenclatures used in connection with techniques of biochemistry, enzymology, molecular and cellular biology, microbiology, genetics, protein and nucleic acid chemistry and hybridization, described herein, are those well-known and commonly used in the art.

Conventional methods and techniques mentioned herein are explained in more detail, for example, in Green & Sambrook (Molecular Cloning, a laboratory manual [fourth edition] Green & Sambrook, Cold Spring Harbor Laboratory, 2012).

A first aspect of the present disclosure provides a modified Saccharomyces cerevisiae cell that differs from an unmodified cell in that it comprises an adhE polypeptide having the amino acid sequence SEQ ID NO: 1, or a functional variant thereof capable of catalysing the conversion of acetyl CoA to ethanol. In contrast to enzymes found naturally in S. cerevisiae, this single enzyme is able to catalyse the formation of ethanol from acetyl CoA. In S. cerevisiae not according to the invention, these activities are present in separate proteins that are localised and regulated differently—five aldehyde dehydrogenases (ALD2 to 6) which can convert acetyl-CoA to acetaldehyde (although their normal activity is the conversion of acetaldehyde to acetyl-CoA) and six alcohol dehydrogenases (ADH1 to 6) which convert acetaldehyde to ethanol.

A second aspect of the present disclosure provides a modified S. cerevisiae cell that differs from an unmodified cell in that it has increased expression of an ACS2 polypeptide having the amino acid sequence SEQ ID NO:2, or a functional variant thereof capable of catalysing the conversion of acetate to acetyl CoA, wherein the unmodified cell is not a sake yeast strain, in particular Kyokai no. 7 or Kyokai no. 701 (Akamatsu et al. (2000) J. Biosci. Bioeng. 90 555-560).

The term “unmodified” cell, as used in this context and throughout this specification, indicates a cell which differs from the modified cell according to the invention only in that it does not include an adhE polypeptide or functional variant and/or does not overexpress an ACS2 polypeptide or functional variant. It does not necessarily indicate a cell which can be found in nature. For example, an “unmodified” cell might be one which is the industrial yeast strain Thermosacc® dry (Lallemand Ethanol Technology, Milwaukee, Wis., USA). In that case, a cell according to the invention would be a Thermosacc® dry cell comprising an adhE polypeptide or functional equivalent and/or overexpressing an ACS2 polypeptide or functional variant thereof. Likewise, the “unmodified” cell may be a S. cerevisiae cell other than Thermosacc® dry, with the cell of the invention being a cell of that strain comprising an adhE polypeptide or functional equivalent, as defined herein.

A modified cell may be a recombinant cell, for example. Typically this may be a cell comprising one or more polynucleotide and/or polypeptide sequences not found within an unmodified cell, as described throughout this specification.

The cell according to the invention may be one which is a combination of the first and second aspects, in that it comprises an adhE polypeptide having the amino acid sequence SEQ ID NO: 1, or a functional variant thereof, and further comprises increased expression of an ACS2 polypeptide having the amino acid sequence SEQ ID NO:2, or a functional variant thereof, compared to the expression in the unmodified cell. In this context, the unmodified cell is one which neither comprises an adhE polypeptide, nor has increased ACS2 expression.

A cell comprising both an adhE polypeptide and an ACS2 polypeptide may utilise acetate in the formation of ethanol by the following pathway:

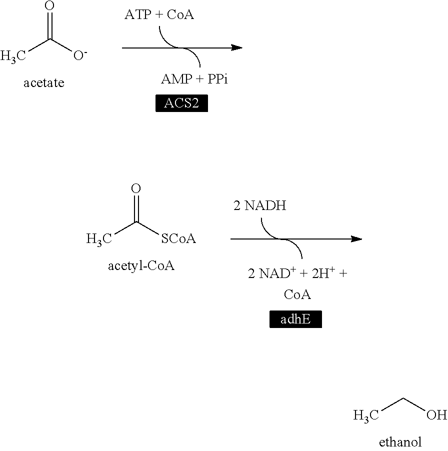

In this specification, SEQ ID NO: 1 provides the amino acid sequence of the enzyme adhE (alcohol/aldehyde dehydrogenase) from Piromyces sp. E2 as expressed in yeast. SEQ ID NO: 2 provides the amino acid sequence of the S. cerevisiae enzyme ACS2 (acetyl-CoA synthetase). SEQ ID NO: 3 is a yeast codon-optimised DNA sequence encoding SEQ ID NO:1; SEQ ID NO:4 is a yeast codon-optimised DNA sequence encoding SEQ ID NO:2. The terms “polynucleotide”, “polynucleotide sequence” and “nucleic acid sequence” are used interchangeably herein. The terms “polypeptide”, “polypeptide sequence” and “amino acid sequence” are, likewise, used interchangeably herein.

A functional variant of an adhE polypeptide having SEQ ID NO:1 is one which forms an enzyme retaining the capability to catalyse the conversion of acetyl CoA to ethanol i.e., an enzyme which can be classified as belonging to both enzyme classes EC 1.2.1.10 and EC 1.1.1.1. This might be tested, for example, by use of the assays described by Boxma et al. (Mol. Microbiol. (2004) 51 1389-1399) or Peng et al. (Anaerobe (2008) 14 125-127), conducted on a sample of isolated enzyme or cell free extract from the yeast strain producing adhE. The functional variant may have an amino acid sequence at least about 50% identical to SEQ ID NO: 1, determined using a Needleman-Wunsch Global Sequence Alignment described below. For example, the functional variant may have sequence identity to SEQ ID NO:1 of at least about 55%, 60%, 65%, 70%, 75%, 80%, 85%, 90%, 91%, 92%, 93%, 94%, 95%, 96%, 97%, 98% or at least about 99%.

A functional variant of an ACS2 polypeptide having SEQ ID NO: 2 is one which forms an enzyme retaining the capability to catalyse the conversion of acetate to acetyl CoA, i.e., an enzyme which can be classified as belonging to enzyme class EC 6.2.1.1. This might be tested, for example, using the assay described Martinez-Blanco et al. (1992; J. Biol. Chem. 267 5474-5481). The functional variant may have an amino acid sequence at least about 50% identical to SEQ ID NO:2, determined using a Needleman-Wunsch Global Sequence Alignment described below. For example, the functional variant may have sequence identity to SEQ ID NO:1 of at least about 55%, 60%, 65%, 70%, 75%, 80%, 85%, 90%, 91%, 92%, 93%, 94%, 95%, 96%, 97%, 98% or at least about 99%.

Enzyme Commission (EC) numbers (also called “classes” herein), referred to throughout this specification, are according to the Nomenclature Committee of the International Union of Biochemistry and Molecular Biology (NC-IUBMB) in its resource “Enzyme Nomenclature” (1992, including Supplements 6-17) available, for example, at http://www.chem.qmul.ac.uk/iubmb/enzyme/. This is a numerical classification scheme based on the chemical reactions catalysed by each enzyme class (Enzyme nomenclature 1992: recommendations of the Nomenclature Committee of the International Union of Biochemistry and Molecular Biology on the nomenclature and classification of enzymes. Webb, E. C. (1992). San Diego: Published for the International Union of Biochemistry and Molecular Biology by Academic Press. ISBN 0-12-227164-5. The skilled person is readily able to determine whether an enzyme falls within any particular enzyme class as referred to herein, for example using methods obtainable from IUBMB.

The cell according to the invention typically has an increased ability to grow (i.e., reduced growth inhibition) on or in a medium containing at least about 50 mM acetate, for example between 50-100 mM acetate, for example at least about 60 mM, 65 mM, 70 mM, 75 mM, 80 mM, 85 mM, 90 mM or at least about 95 mM, or up to 100 mM.

The increased ability to grow may indicate an increased number of colonies when a sample comprising the cell is cultured on a solid medium containing at least about 50 mM acetate, for example on a solid medium containing at least about 75 mM, 80 mM or 90 mM acetate. The medium may be a YPD (Yeast peptone dextrose) medium or a Synthetic Complete medium, as described herein. Culturing a sample comprising the cell may involve routine microbiology techniques, for example starting a culture by applying a small amount of a liquid culture of the cell to the surface of an agar plate comprising the medium and storing the plate under appropriate conditions, for example at about 30° C. for at least about 12 hours, for example for 12-168 hours, for example 15-50 hours, for example for about 24, 36 or 48 hours. Such techniques are routine for the skilled person and are also described herein.

Alternatively or additionally, the increased ability to grow may indicate an increase in OD₆₀₀ when a sample comprising the cell is cultured in a liquid medium containing at least about 50 mM acetate, for example on in a liquid medium containing at least about 75 mM, 80 mM or 90 mM acetate. The medium may be a YPD medium or a Synthetic Complete medium, as described herein. Culturing a sample comprising the cell may involve routine microbiology techniques, for example starting a culture by adding a small amount of an overnight liquid culture of the cell to a fresh liquid culture medium and storing the resulting mixture under appropriate conditions, for example at about 30° C. with agitation of about 250 rpm, for at least about 12 hours, for example for 12-168 hours, for example 15-50 hours, for example for about 24, 36 or 48 hours. Such techniques are routine for the skilled person and are also described herein.

In the solid and liquid media mentioned above, the acetate may be provided by the presence in the medium of sodium acetate and/or acetic acid in the medium. The pH of the solid or liquid media may be pH 3.5-7, for example pH 4-5, for example about pH 4.5. Where the acetate is provided by the presence of sodium acetate, the pH may be about 6.5.

The increase in OD₆₀₀ may be an increase of at least about 20%, for example, at least about 25%, 30%, 40%, 50%, 60%, 70%, 80%, 90% or 100%. For example, where the cell comprises an adhE polypeptide or functional variant, the increase may be 20-30%, for example about 25%. Where the modified cell comprises increased expression of an ACS2 polypeptide or functional variant, the increase may be 70-75%, for example, about 70% or about 71%. Where the modified cell comprises an adhE polypeptide or functional variant and increased expression of an ACS2 polypeptide or functional variant, the increase may be 50-55%, for example about 51%, 52% or about 53%. In all of these examples, the medium may comprise about 75 mM acetate.

Alternatively or additionally, the increased ability to grow may indicate an increased rate of survival when a sample comprising the cell is cultured on a solid medium containing at least about 50 mM acetate, for example on a solid medium containing at least about 75 mM, 80 mM or 90 mM acetate. The medium may be a YPD medium or a Synthetic Complete medium, as described herein. Culturing a sample comprising the cell may involve routine microbiology techniques, as described above and elsewhere herein. Survival rate may assessed, for example, by dispensing aliquots of serial dilutions (for example, serial 10-fold dilutions) of a liquid sample comprising the cell to the surface of a solid medium and observing the number of colonies which grow. Survival rate may be assessed, for example, at least 15-20 hours, for example about 20 h, 24 h, 36 h or about 48 h after starting the culture of the sample. A more detailed description of this technique is described in the Examples section below. When the medium contains about 75 mM acetate, the rate of survival may be increased by up to about 100-fold, where the rate of survival may be about 100% cell survival. When the medium contains about 90 mM acetate, the rate of survival may be increased by up to about 10-fold; for example, the rate of survival may be about 1% cell survival.

The cell according to the invention may have increased glucose utilisation over at least about 20 hours in comparison to a unmodified cell, for example over about 24 hours. This may be assessed by obtaining a sample from a liquid culture of the cell (as described above and elsewhere herein) and carrying out High Performance Liquid Chromatography (HPLC) on the sample, to determine the amount of glucose in the medium in which the cell has been grown compared to a sample of equivalent media in which a unmodified cell sample has grown over the same period.

Alternatively or additionally, the cell according to the invention may have increased ethanol production over at least about 20 hours in comparison to a unmodified cell, for example over about 24 hours. The increased ethanol production may be at least about 40%, 45% or at least about 50% increased production over this period, compared to a unmodified cell not according to the invention.

The amount of glucose and/or ethanol present at the end of the time period may be determined using HPLC as described above, with a comparison optionally being made to the amount of ethanol present in the medium at 0 hours, i.e., immediately after a culture has been started by adding a cell sample to fresh culture medium.

The modified cell according to the invention may be a recombinant cell.

The modified cell according to the invention may comprise a polynucleotide sequence encoding SEQ ID NO:1 or encoding a functional equivalent thereof, as described above. For example, the polynucleotide sequence may be SEQ ID NO: 3.

Alternatively or additionally the cell may comprise a polynucleotide sequence encoding SEQ ID NO:2 or encoding a functional equivalent thereof, as described above. For example, the polynucleotide sequence may be SEQ ID NO: 4.

The polynucleotide may be contained in an expression vector (which may be one of, for example, SEQ ID NOs: 14-19, 22, 23 or 24) and/or may be integrated into the cell genome, for example by integration into the S. cerevisiae HO gene. Therefore, the cell may have a genome comprising at least one polynucleotide sequence encoding either or both the enzymes adhE and/or ACS2, such as a polynucleotide having sequence SEQ ID NO: 3 or 4 or a functional variant or portion thereof. For example, the cell may have a genome comprising construct SEQ ID NO: 20 or 21.

Advantages of preparing a modified cell according to the invention by means of inclusion in the cell genome of a polynucleotide encoding the enzyme may include the stable inheritance of the polynucleotide. This may remove any need to carry out constant selection in order to maintain a vector. Furthermore, incorporation of a polynucleotide into the genome may ensure a stable copy number, whereas vector copy number can fluctuate in cultures.

The present invention also encompasses a cell comprising functional variants or portions of the enzyme, as described above. As used herein, a “variant” means an enzyme in which the amino acid sequence differs from the base sequence from which it is derived in that one or more amino acids within the sequence are substituted for other amino acids, or deleted. For example, a variant of SEQ ID NO:1 may have an amino acid sequence at least about 50% identical to SEQ ID NO:1, for example, at least about 55%, 60%, 65%, 70%, 75%, 80%, 85%, 90%, 91%, 92%, 93%, 94%, 95%, 96%, 97%, 98%, 99% or about 100% identical. This may be the consequence of substitutions or deletions of amino acids within the original sequence SEQ ID NO:1. The variants or portions are functional variants or portions in that the variant sequence has similar or, preferably, identical functional enzyme activity characteristics to the enzyme having the non-variant amino acid sequence (and this is the meaning of the term “functional variant or portion” or the term “functionally equivalent” as used throughout this specification).

A functional variant may be a homologue of SEQ ID NO:1 found in species other than Piromyces sp. E2, whilst a homologue of SEQ ID NO:2 may be found in species or strains other than the S. cerevisiae strains described herein. Determining homologues is within the routine ability of the skilled person, utilising software such as that available from NCBI mentioned below.

The similar or identical enzyme characteristics as SEQ ID NO:1 (for example) may be assessed, for example, by comparing the rate of conversion of acetyl-CoA to ethanol by a variant, to the rate achieved by SEQ ID NO:1 itself. For a functional variant, this rate may be the same or similar, for example at least about 60%, 70%, 80%, 90%, 95% or about 100% the rate achieved by SEQ ID NO:1. A functional variant may also have greater (i.e., an improved) activity than that of SEQ ID NO:1, i.e., the rate of conversion of acetyl-CoA to ethanol may be more than 100% compared to that of SEQ ID NO:1. Equivalent determinations may be made regarding the rate of conversion of acetate to acetyl CoA by the enzyme with sequence SEQ ID NO:2. Suitable methods for determining the enzyme activity are outlined above.

Amino acid substitutions may be regarded as “conservative” where an amino acid is replaced with a different amino acid with broadly similar properties. Non-conservative substitutions are where amino acids are replaced with amino acids of a different type.

By “conservative substitution” is meant the substitution of an amino acid by another amino acid of the same class, in which the classes are defined as follows:

| Class | Amino acid examples |
|---|---|
| Nonpolar: | A, V, L, I, P, M, F, W |
| Uncharged polar: | G, S, T, C, Y, N, Q |
| Acidic: | D, E |
| Basic: | K, R, H |

As is well known to those skilled in the art, altering the primary structure of a polypeptide by a conservative substitution may not significantly alter the activity of that polypeptide because the side-chain of the amino acid which is inserted into the sequence may be able to form similar bonds and contacts as the side chain of the amino acid which has been substituted out. This is so even when the substitution is in a region which is critical in determining the polypeptide's conformation.

In the present invention, non-conservative substitutions are possible provided that these do not interrupt the enzyme activities of the polypeptides, as defined elsewhere herein.

Broadly speaking, fewer non-conservative substitutions than conservative substitutions will be possible without altering the biological (i.e., enzymatic) activity of the polypeptides. Determination of the effect of any substitution (and, indeed, of any amino acid deletion or insertion) is wholly within the routine capabilities of the skilled person, who can readily determine whether a variant polypeptide retains the activity of the non-variant enzyme, as discussed above.

Using the standard genetic code, further nucleic acid sequences encoding the polypeptides may be readily conceived and manufactured by the skilled person, in addition to those disclosed herein. The nucleic acid sequence may be DNA or RNA and, where it is a DNA molecule, it may for example comprise a cDNA or genomic DNA. The nucleic acid may be contained within an expression vector, such as a plasmid as described elsewhere herein.

The invention, therefore, encompasses use of variant nucleic acid sequences encoding the polypeptides of the invention. The term “variant” in relation to a nucleic acid sequence means any substitution of, variation of, modification of, replacement of, deletion of, or addition of one or more nucleic acid(s) from or to a polynucleotide sequence, providing the resultant polypeptide sequence encoded by the polynucleotide exhibits at least the same or similar enzymatic properties as the polypeptide encoded by the basic sequence. The term therefore includes allelic variants and also includes a polynucleotide (a “probe sequence”) which substantially hybridises to the polynucleotide sequences disclosed herein. Such hybridisation may occur at or between low and high stringency conditions. In general terms, low stringency conditions can be defined as hybridisation in which the washing step takes place in a 0.330-0.825 M NaCl buffer solution at a temperature of about 40-48° C. below the calculated or actual melting temperature (Tₘ) of the probe sequence (for example, about ambient laboratory temperature to about 55° C.), while high stringency conditions involve a wash in a 0.0165-0.0330M NaCl buffer solution at a temperature of about 5-10° C. below the calculated or actual Tₘ of the probe sequence (for example, about 65° C.). The buffer solution may, for example, be SSC buffer (0.15M NaCl and 0.015M tri-sodium citrate), with the low stringency wash taking place in 3×SSC buffer and the high stringency wash taking place in 0.1×SSC buffer. Steps involved in hybridisation of nucleic acid sequences have been described for example in Green & Sambrook (as above).

Typically, nucleic acid sequence variants have about 55% or more of the nucleotides in common with the nucleic acid sequence of the present invention, more typically about 60%, 65%, 70%, 80%, 85%, or even 90%, 95%, 98% or about 99% or greater sequence identity.

Variant nucleic acids may be codon-optimised for expression in a particular host cell.

Sequence identity between amino acid and nucleic acid sequences is determined herein (for both patentability and infringement considerations) by comparing an alignment of the sequences using the Needleman-Wunsch Global Sequence Alignment Tool available from the National Center for Biotechnology Information (NCBI), Bethesda, Md., USA, for example via http://blast.ncbi.nlm.nih.gov/Blast.cgi, using default parameter settings (for protein alignment, Gap costs Existence: 11 Extension:1). Sequence comparisons and percentage identities mentioned in this specification have been determined using this software. When comparing the level of sequence identity to, for example, SEQ ID NO:1, this typically should be done relative to the whole length of SEQ ID NO:1 (i.e., a global alignment method is used). When an equivalent position in the compared sequences is occupied by the same amino acid, then the molecules are identical at that position. Scoring an alignment as a percentage of identity is a function of the number of identical amino acids at positions shared by the compared sequences. When comparing sequences, optimal alignments may require gaps to be introduced into one or more of the sequences, to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties. As mentioned above, the percentage sequence identity may be determined using the Needleman-Wunsch Global Sequence Alignment tool, using default parameter settings. The Needleman-Wunsch algorithm was published in J. Mol. Biol. (1970) vol. 48:443-53.

A third aspect of the invention provides a method of improving the ability of a yeast cell to grow on a solid medium, or in a liquid medium, the medium comprising at least about 50 mM acetate, wherein the method comprises:

a) introducing to the cell an adhE polypeptide and/or a functional variant thereof; and/or

b) increasing expression of an ACS2 polypeptide and/or a functional variant thereof.

The term “increasing expression” indicates that the cell which is the subject of the method displays an increased amount of the ACS2 polypeptide in comparison to an equivalent (i.e., otherwise identical) cell which is not the subject of the method.

The cell may be a S. cerevisiae cell, for example, a Thermosacc® dry cell. The cell may be one which is not of strain Kyokai no. 7 or Kyokai no. 701.

The method may comprise transforming the cell with a polynucleotide encoding SEQ ID NO: 1 or a functional equivalent thereof and/or a polynucleotide encoding SEQ ID NO:2 or a functional equivalent thereof. For example, the polynucleotide may have sequence SEQ ID NO:3 or 4. The polynucleotide may be contained within a vector and/or may become integrated into the cell genome, for example in the S. cerevisiae HO gene.

In the method, the improved ability of the yeast cell to grow in a liquid medium comprising at least about 75 mM acetate may be at least about 25% increased growth compared to an equivalent cell not comprising adhE and/or ACS2. For example, this may be assessed by determining the OD₆₀₀ of a liquid culture of the cell, for example, at 15-30 hours, e.g., about 24 h, 36 h or about 48 h after the liquid culture is started. Where the cell comprises an adhE polypeptide or functional variant, the increase may be 20-30%, for example about 25%. Where the cell comprises increased expression of an ACS2 polypeptide or functional variant, the increase may be 70-75%, for example, about 70% or about 71%. Where the cell comprises an adhE polypeptide or functional variant and increased expression of an ACS2 polypeptide or functional variant, the increased growth may be 50-55%, for example about 51%, 52% or about 53%.

In the method, where an ACS2 polypeptide or functional equivalent is overexpressed in the cell, or where an ACS2 polypeptide or functional equivalent is overexpressed in the cell and an adhE polypeptide or functional equivalent is expressed in the cell, the improved ability to grow on a solid medium comprising at least about 75 mM acetate may be about 100% cell survival. The improved ability to grow may be an improved ability to grow on a solid medium comprising at least about 90 mM with the improved ability being a 10-fold increase in the cell survival, for example about 1% cell survival. This may be assessed, for example, at 15-30 hours, e.g., about 24 h, 36 h or about 48 h after the liquid culture is started.

According to a fourth aspect of the invention, there is provided a method of increasing ethanol yield from a yeast cell culture within up to about 30 hours from starting the culture, comprising:

a) overexpressing an ACS2 polypeptide and/or a functional variant thereof in the cells of the culture; and/or

b) comprising expressing an adhE polypeptide and/or a functional variant thereof in the cells;

culturing the cell in or on a medium comprising glucose and determining ethanol yield up to about 30 hours from the starting of the culture. The medium may further comprise acetate, which may be utilised by the cell in the formation of ethanol.

The increase in ethanol yield may be observable, for example by use of HPLC as described elsewhere herein, at about 24 hours after the formation or starting of the culture, i.e., about 24 hours after a sample comprising the cells modified by the method is added to a new sample of liquid medium. The increase may be in comparison to the ethanol yield from a sample of cells which have not been modified by the method. The increase may be at least about 40%, 45% or at least about 50% compared to the non-adapted cells.

The method according to the fourth aspect of the invention may subsequently comprise isolating the alcohol.

A fifth aspect of the invention provides a method for the production of a biofuel and/or a biochemical comprising combining an alcohol produced by a cell according to the first or second aspects of the invention and/or in a method according to the third aspect of the invention with one or more additional components to produce a biofuel and/or biochemical.

The alcohol can be blended as a biofuel component and/or a biochemical component with one or more other components to produce a biofuel and/or a biochemical. By a biofuel or a biochemical, respectively, is herein understood a fuel or a chemical that is at least partly derived from a renewable energy source. Examples of one or more other components with which the alcohol may be blended include anti-oxidants, corrosion inhibitors, ashless detergents, dehazers, dyes, lubricity improvers and/or mineral fuel components, but also conventional petroleum derived gasoline, diesel and/or kerosene fractions.

A sixth aspect of the invention provides the use of a cell according to the first aspect of the invention as a biofuel/biochemical alcohol source. A “biofuel/biochemical alcohol” is an alcohol (such as ethanol) or mixture of alcohols, which may be used in the preparation of a biofuel and/or a biochemical, for example in a method according to the fifth aspect of the invention. The use of a cell as the source of such a precursor indicates that the cell according to the first or second aspects of the invention produces alcohol suitable for use in the biofuel/biochemical production methods, the alcohol being isolatable from the cell as described elsewhere herein.

In some embodiments, there is provided a modified Saccharomyces cerevisiae cell that differs from a unmodified cell in that it comprises an adhE polypeptide having the amino acid sequence SEQ ID NO:1, or a functional variant thereof capable of catalysing the conversion of acetyl CoA to ethanol. In one embodiment, the functional variant has an amino acid sequence at least about 50% identical to SEQ ID NO:1.

In other embodiments, there is provided a modified Saccharomyces cerevisiae cell that differs from a unmodified cell in that it has increased expression of an ACS2 polypeptide having the amino acid sequence SEQ ID NO:2, or a functional variant thereof capable of catalysing the conversion of acetate to acetyl CoA, wherein the unmodified cell is not the sake yeast strain Kyokai no. 7 or the strain Kyokai no. 701.

In one embodiment, a cell according to any preceding claim comprising an adhE polypeptide having the amino acid sequence SEQ ID NO:1, or a functional variant thereof, further comprises increased expression of an ACS2 polypeptide having the amino acid sequence SEQ ID NO:2, or a functional variant thereof, compared to the expression in the unmodified cell. In another embodiment, a cell according to embodiments described above has an increased ability to grow on or in a medium containing at least about 50 mM acetate. In one embodiment, the increased ability to grow indicates an increased number of colonies when a sample comprising the cell is grown on solid media containing at least about 50 mM acetate. In one embodiment, the increased ability to grow indicates an increased in OD₆₀₀ when a sample comprising the cell is grown in liquid media containing at least about 50 mM acetate. In one embodiment, the increased ability to grow indicates an increased rate of survival when a sample comprising the cell is grown on solid media containing at least about 50 mM acetate. In one embodiment, the unmodified cell is a Thermosacc® dry cell. In one embodiment, the acetate is provided by the presence of sodium acetate and/or acetic acid in the medium. In one embodiment, the pH of the solid or liquid media is pH 4-5. In another embodiment, the acetate is provided by the presence of sodium acetate and the pH is about 6.5.

In some embodiments, a cell according to embodiments described above has increased glucose utilisation over at least about 20 hours in comparison to an unmodified cell. In other embodiments, a cell according to embodiments described above has increased ethanol production over at least about 20 hours in comparison to an unmodified cell. In one embodiment, the increased ethanol production is an increase of at least about 40%.

In some embodiments, the cell comprises a polynucleotide sequence encoding SEQ ID NO:1 or encoding a functional equivalent thereof. In one embodiment, the polynucleotide sequence is SEQ ID NO:3. In some embodiments, the cell comprises a polynucleotide sequence encoding SEQ ID NO:2 or encoding a functional equivalent thereof. In one embodiment, the polynucleotide sequence is SEQ ID NO:4. In one embodiment, the polynucleotide is contained in an expression vector and/or is integrated into the cell genome.

In some embodiments, there is provided a method of improving the ability of a yeast cell to grow on a solid medium, or in a liquid medium, the medium comprising at least about 50 mM acetate, wherein the method comprises: (a) introducing to the cell an adhE polypeptide and/or a functional variant thereof; and/or (b) increasing expression of an ACS2 polypeptide and/or a functional variant thereof. In one embodiment, the cell is a S. cerevisiae cell. In another embodiment, the cell is a Thermosacc® dry cell.

The method according to any of claims 21-23 comprising transforming the cell with a polynucleotide encoding SEQ ID NO:1 and/or SEQ ID NO:2. On one embodiment, the polynucleotide has sequence SEQ ID NO:3 or 4. In one embodiment, the polynucleotide is contained within a vector and/or wherein the polynucleotide becomes integrated into the cell genome.

In one embodiment, the improved ability of the yeast cell to grow in a liquid medium comprising at least about 75 mM acetate is at least about 25% increased growth compared to an equivalent cell not comprising adhE and/or ACS2 or a functional equivalent thereof. In another embodiment, an ACS2 polypeptide or functional variant is overexpressed in the cell, or an ACS2 polypeptide or functional variant is overexpressed in the cell and an adhE polypeptide or functional variant is introduced into the cell, and the improved ability to grow on a solid medium comprising at least about 75 mM acetate is about 100% cell survival.

In some embodiments, there is provided a method of increasing ethanol yield within up to about 30 hours from a yeast cell, comprising: (a) overexpressing an ACS2 polypeptide and/or a functional variant thereof in the cell; and/or (b) comprising expressing an adhE polypeptide and/or a functional variant thereof in the cell; and (c) culturing the cell in or on a medium comprising glucose. In one embodiment, the increase in ethanol yield is an increase of at least about 40% compared to the yield obtained from a unmodified cell. In another embodiment, the medium further comprises acetate. In yet another embodiment, the cell utilises the acetate in the formation of ethanol.

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

### BRIEF DESCRIPTION OF THE DRAWINGS

Embodiments of the invention will now be described, by way of example only, with reference to the following FIGS. 1-9 in which:

FIG. 1 shows the results of a spot assay of 10-fold serial dilutions (left to right) on YPD plates, showing that expression of either ACS2 or adhE increases tolerance of Thermosacc® dry to acetate;

FIG. 2 shows that expression of ACS2 or adhE increases growth, glucose consumption and ethanol production after 24 hours in 75 mM acetate, with dotted lines showing upper and lower limits of the range of values obtained for the control strain 5491;

FIG. 3 is a Western blot showing production of the adhE and ACS2 proteins after 24 hours in YPD medium (2% glucose) alone (−), with 75 mM acetate (ac) and 2% ethanol (etOH);

FIG. 4 shows the results of a spot assay of 10-fold serial dilutions (left to right) on YPD plates, showing that expression of either ACS2 or adhE increases tolerance of Thermosacc® dry to 75 mM acetic acid (B) and 75 mM sodium acetate (C), with (A) being YPD plates containing clonNAT™ alone;

FIG. 5 shows chromosomal integration constructs for ACS2 and adhE;

FIG. 6 shows the results of a spot assay of 10-fold serial dilutions (left to right) on YPD plates, showing that integration of an ICL1 promoter-regulated ACS2 gene increases acetate tolerance of Thermosacc® dry, assays being done in duplicate on YPD plates and grown for 24 hours (A), or YPD supplemented with 75 mM acetic acid (B) or 90 mM acetic acid (C), cultures on acetate-containing plates being grown for 40 hours;

FIG. 7 shows the results of a spot assay of 10-fold serial dilutions (left to right) on YPD plates, showing that integration of ACS2 with or without adhE increases acetic acid tolerance at pH 4.5, but not tolerance to sodium acetate at pH6.5, with (A) showing growth of serially diluted strains after 24 hours on YPD alone, YPD plus 75 mM acetic acid (pH 4.5) and YPD plus 75 mM sodium acetate (pH 6.5) and (b) showing growth of serially diluted strains after 48 hours on YPD plus 90 mM acetic acid (pH 4.5), YPD plus 75 mM acetic acid (pH 4.5) and YPD plus 75 mM sodium acetate (pH 6.5);

FIG. 8 shows growth, acetate and ethanol levels of plasmid-bearing strains adhE (S528) and ACS2 (S564) in cultures grown in Synthetic Complete (SC) medium, with either 1% glucose given as sole carbon source (left side) or 75 mM sodium acetate added to SC with 1% glucose; the figure shows time course of biomass production (A), acetate production and utilisation (B) and ethanol production (C); and

FIG. 9 shows growth and acetate levels in cultures where sodium acetate was given as a sole carbon source, with WT Thermosacc® dry and the ACS2 integration strain 5587 shown on the left and the plasmid-bearing strains control (S485), adhE (S528) and ACS2 (5564) shown on the right; the figure shows (A) time course of growth of ACS2 integration strains (left) and plasmid strains (right) in SC medium with 50 mM sodium acetate and (B) time course of acetate utilisation.

### EXAMPLES

Yeast strains provided by the following examples are summarised in Tables 5, 6 and 7 below. Plasmids and DNA constructs provided by the examples are summarised in Table 9 below.

### Yeast YPD (Yeast Peptone Dextrose) Plates

Plates were made up with using the medium shown in Table 1:

TABLE 1
components of YPD medium:

| Ingredient | Concentration |
|---|---|
| yeast extract | 10 g/L |
| bacto peptone | 20 g/L |
| D-glucose | 20 g/L |
| agar | 20 g/L |

Antibiotics for plasmid selection and inducers of gene expression (as indicated in both Table 2 and Table 4), were added when molten YPD was cooled to 50° C., before pouring plates. pH was not adjusted after adding acetate, but was measured as >pH4 for all acetate plates made.

TABLE 2
concentrations of media components

| | |
|---|---|
| | Stock |
| Final | Concentration |
| Concentration | in Water |

antibiotics for selection

| | | | |
|---|---|---|---|
| clonNAT selection for natMX | 100 | μg/ml | 200 mg/ml (2000X) |
| marker (nourseothricin; source: | | | |
| Werner Bioagents) | | | |
| G418 selection for kanMX | 200 | μg/ml | 200 mg/ml (1000X) |
| marker(source: Sigma) | | | |

inducers

| | | | |
|---|---|---|---|
| CuSO4 (source: Fisons | 250 | μM | 200 mM (800X) |
| Scientific) | | | |
| doxycycline (source: Sigma) | 5 | μg/ml | 10 mg/ml (2000X) |

acetate

| acetate (source: Sigma) | 50, 75, 100 mM | | glacial acetic acid, |
|---|---|---|---|
| | | | 17.41M |
| Sodium acetate anhydrous | 75 | mM | Stock 2.5M in water |
| (source: Riedel de Haën) | | | |

### Yeast YPD Liquid Media

Liquid media was made to the same recipe as the plates above, but lacking agar. All inducers and antibiotics were added at the same concentrations as for the plates above. pH adjustments were not carried out but the following measurements were made:

YPD without acetate: pH 6.11;

YPD with 50 mM acetate, with all antibiotics and inducers:

pH 4.38;

YPD with 75 mM acetate, with all antibiotics and inducers:

pH 4.23.

### Construction of Empty Vectors

Two empty vector control plasmids were engineered. pS452 (Table 9) based on the pRS416 plasmid (Genbank ID U03450.1; Sikorski and Hieter (1989) Genetics, 122 19-27). The URA3 marker on pRS416 was replaced with the natMX antibiotic marker by gap repair, as follows. pRS416 was digested at the URA3 locus with NcoI and then 100 ng of this digested plasmid was co-transformed with 100 ng of PCR-amplified natMX gene. The natMX gene was amplified with primers pRSnat-switch-F and pRSnat-switch-R (Table 8) and template plasmid pAG25 (Goldstein & McCusker (1999) Yeast 15, 1541-15). This PCR reaction and all others (unless otherwise stated) were done using KOD Polymerase (Novagen). Yeast transformation protocol is described below. Transformants were selected on YPD plates with clonNAT antibiotic (concentration shown on Table 2). Successful gap repair was verified by colony PCR using DreamTaq polymerase (Thermo Fisher) and primers pRS-check-switch-R and natC (Table 8) which produced a 479 bp DNA product. pS453 (Table 9) is based on pCM251 (Bellí et al. (1998) Nuc. Ac. Res. 26 U8-U8) which contains the tetO2 promoter and a transcription factor that is responsive to the antibiotic doxycycline. The TRP1 marker in pCM251 was replaced with the kanMX marker by gap repair, as follows. pCM251 was digested at the TRP1 locus with EcoRV. 100 ng of digested plasmid was then co-transformed with 100 ng of PCR-amplified kanMX gene (using primers CM251sw-F and CM251sw-R (Table 8) and template plasmid pFA6 (Wach et al. (1994) Yeast, 10 1793-1808) into the yeast Thermosacc® dry. Transformants were selected on YPD medium containing G418 (concentration shown in Table 2). Gap repair was verified by colony PCR using primers CM251-gap-F and pTEF-R (Table 8), giving a DNA product of 553 bp.

To rescue plasmids from yeast, transformant colonies were grown in liquid YPD with the appropriate antibiotic. Cell pellets from 1 ml of overnight cultures were lysed in 50 μl Cell Resuspension Solution (Promega SV Mini Kit) with agitation with 50 μl of 0.5 mm glass beads (Stratech) for a total of 2 minutes at 5 m/s in a Fast Prep (MP Bio) bead beater. DNA was isolated using the SV Mini Kit (Promega), of which 31 μl was used to transform into competent DH10-beta E. coli cells. Transformants were selected on Luria-Bertani agar plates with 100 μg/ml carbenicillin (Fisher Scientific) and subsequently grown to isolate the DNA plasmids.

Construction of Plasmid with ACS2 Under the TetO2 Promoter

ACS2 gene was amplified from genomic DNA of S. cerevisiae BY4741 strain (Brachmann et al. (1998) Yeast 14 115-132). The primers used were designed with the restriction enzyme sites NotI and PstI at the N- and C-termini, respectively (using primers NotACS2-F and PstBclACS2-R, Table 8). The amplified gene was cut with NotI and PstI and ligated into pS446 (also digested with NotI and PstI for this ligation; this vector is based on pCM251 and its sequence is provided as SEQ ID NO:22). The resulting plasmid, pCM251 with a hexahistidine-tagged ACS2 gene, was then modified to carry the kanMX marker. This was done by gap repair, as described above for creating the 5453 empty vector, the resulting plasmid is pS453-ACS2.

Construction of Plasmid with AdhE Under the CUP1 Promoter

Piromyces sp. E2 AdhE (UniProt ID Q6WJD5; Boxma et al. (2004) Mol. Microbiol. 51 1389-1399) was codon-optimized for S. cerevisiae, designed with an N-terminal streptavidin tag and the NdeI and BamHI restriction sites at its N- and C-termini, respectively, and synthesized (GeneArt). The digested AdhE fragment was then ligated into NdeI and BamHI digested pS440 (the sequence of which is provided as SEQ ID NO:24), resulting in plasmid pS465.

It was not possible to detect production of the N-terminal tagged AdhE protein from pS465, so the C-terminal tagged version was created as follows. AdhE was amplified in two parts and two rounds of PCR (N-terminal 1-1425 bp and C-terminal 1077-2655 bp which added C-terminal strep tag). For the first round of PCR for the N-terminal part, the original N-tagged AdhE sequence was used as template with primers adhE-Nde-Cstrep-F and adhE-1425-R (Table 8). 80 bp of homology was added to the CUP1 promoter by using 10 to 20 ng of the first PCR product as template for a second PCR using primers CupP-extender-F and adhE-1425-R. For the first round of PCR for the C-terminal part, the original N-tagged AdhE sequence was used as template with primers adhE-1077-F and adhE-Cstrep-Bam R (Table 8) to add the C-terminal strep tag. 80 bp of homology was added to the CYC1 terminator by using 10 to 20 ng of the first PCR product as template for a second PCR using primers adhE-1077-F and CycT-extender-R. These two fragments have 400 bp of homology between them and were cloned by gap repair into a yeast vector as follows. About 100 ng of each fragment was transformed into Thermosacc® dry with 100 ng of NdeI and BamHI digest pS440. Transformants were selected on YPD with clonNAT antibiotic and were verified by colony PCR using primers T7 and adhE-R (Table 8). The resulting plasmid is pS488, and transformants were subsequently shown to produce the tagged AdhE protein.

### Plasmid Transformation and Gap Repair Protocol

To create the above plasmids by gap repair, as well as transform intact plasmids into Thermosacc® dry, a combination of both high efficiency transformation protocol and quick and easy transformation method using lithium acetate were used (Gietz & Schiestl (2007) Nature Protocols 2 38-41; Gietz & Schiestl (2007) Nature Protocols 2 35-37).

0.5 ml from an overnight liquid culture of Thermosacc® dry in YPD was spun down and the cell pellet was washed in 1 ml of water. Cells were re-pelleted and mixed with 15 μl of 10 mg/ml salmon sperm DNA (Sigma) and DNA of interest (PCR products, digests, intact plasmids).
To this a transformation mix was added, in a total volume 360 μl including DNA and ssDNA:

TABLE 3
transformation mix components

| Ingredient | volume |
|---|---|
| PEG 3350 (Sigma, stock 50% (w/v)) | 240 μl |
| LiAc (Sigma, stock 1.0M) | 36 μl |
| Sterile water | to total 360 μl |

Cells were heat-shocked at 42° C. for 40 minutes and then pelleted. The transformation mix was removed and cells were resuspended in 1 ml YPD for recovery of 2 to 3 hours at 30° C. Cells were pelleted, resuspended in 100 μl water and plated out on YPD with the appropriate antibiotic. Gap repair is a method used to add one or more inserts into a plasmid. In each case, 100 ng of each the digested plasmid and 100 ng each PCR product to be inserted were used. These amounts were the same used by Kitazono (2011; Gene 484 86-89).

### Yeast Strain Cultures and Induction Conditions

Pre-cultures of Thermosacc® dry were grown in YPD with antibiotics clonNAT™ and G418 overnight (final concentrations as in Table 2). New cultures were made in YPD+clonNAT™+G418+CuSO₄+doxycycline (final concentrations as in Table 4)+acetate. The following culture conditions were used:
a. 10 ml cultures with 75 mM acetate (see Table 2) in 50 ml Falcon tubes, grown at 30° C. with 250 rpm shaking. Starting OD₆₀₀ of 0.3.
b. 1.5 ml cultures with 50 mM or 75 mM acetate, in 2 ml screw-cap microfuge tubes, grown at 30° C. with 250 rpm shaking. Starting OD₆₀₀ of approximately 0.01 (started with 15 μl overnight culture).

TABLE 4
Expression of Heterologous Genes

| | | Plasmid | | |
|---|---|---|---|---|
| | Organism and | selection and | Construct | Induction |
| Enzyme | Gene Name | Parent Strain | Details | conditions |
| ACS | S. cerevisiae | 200 μg/ml | N-terminal | 5 μg/ml |
| | ACS2 | G418 in strain | his6 tag; | doxycycline, |
| | size: 683 a.a. | Thermosacc ® | MW 77 kDa | 7 hours |
| | | dry | | |
| adhE | Piromyces | 100 μg/ml | C-terminal | 50-250 |
| | sp. E2 adhE | clonNAT ™ | strep tag; | μg/ml CuSO₄, |
| | size: 885 a.a. | in strain | MW 98 kDa | 2-4 hours |
| | | Thermosacc ® | | |
| | | dry | | |

### Western Blot Analysis

Cultures were grown with the appropriate antibiotic selection for 24 hours and growth was measured using optical density at 600 nm. Total protein was then extracted from 8 OD units of cells (roughly 8×10⁷ cells), using the trichloroacetic acid precipitation method (Paciotti et al (1998) EMBO J. 17 4199-4209). 10 μl of protein extracts were on a 10-20% polyacrylamide gel (Invitrogen) and transferred onto a PVDF membrane. To detect streptavidin-tagged adhE, the blot was incubated for 1 hour with 1:4000 dilution of Strep-Tactin alkaline phosphatase conjugate (IBA). To detect hexahistidine-tagged ACS2, the blot was incubated with 1:2000 dilution of anti-his antibody conjugated with alkaline phosphatase (Sigma).

After washing the membrane, it was incubated in BCIP/NBT substrate solution (Sigma) until distinct bands corresponding to the tagged proteins were visible (10-20 minutes).

AdhE protein (copper-induced) was detectable at 2 to 4 hours after induction. ACS2 protein was detectable at 6 hours. Transformants were visible after 2 to 3 days and were confirmed by colony PCR specific to the ACS2 and AdhE genes. Western blots also confirmed that the expected proteins were produced. AdhE expression of the C-tagged version is visible at 2 and 4 h of induction with copper sulfate. ACS2 expression of the N-tagged version is faintly visible after 7 h or overnight induction with doxycycline. For HPLC (to measure acetate, glucose, ethanol), culture supernatant was collected at 20-24 h.

### Double Transformants

To create double transformants (Strains 5494 and 5495 containing both AdhE and ACS2 plasmids; Table 5), the AdhE plasmid was rescued from yeast as described above and transformed into competent E. coli with ampicillin selection. Approximately 300 ng of miniprepped AdhE plasmid was transformed into the ACS2 strain using the method described above. The ACS2 strain was pre-grown in YPD containing G418 antibiotic and double transformants were selected on YPD plates containing both G418 and clonNAT™. A control strain was also constructed bearing the empty plasmid vectors that would allow it to grow in the presence of the clonNAT™ and G418 antibiotics.

TABLE 5
Engineered yeast strains

| Strain | | |
|---|---|---|
| Name | Genotype | Comments |
| S491 | Thermosacc ® + plasmid pS453 | control strain |
| | (empty vector kanMX) + plasmid pS452 | |
| | (empty vector natMX) | |
| S492 | Thermosacc ® + plasmid pS453-ACS2 | producing ACS2 only |
| | (ACS2-Ntag in pS453) + plasmid pS452 | |
| | (empty vector natMX) | |
| S493 | Thermosacc ® + plasmid pS453 | producing adhE only |
| | (empty vector kanMX) + plasmid pS488 | |
| | (adhE-Ctag in pS452) | |
| S494 | Thermosacc ® + plasmid pS453-ACS2 | producing both |
| | (ACS2-Ntag in pS453) + plasmid pS488 | proteins |
| | (adhE-Ctag in pS453) | |
| S495 | Thermosacc ® + plasmid pS453-ACS2 | producing both |
| | (ACS2-Ntag in pS453) + plasmid pS488 | proteins, independent |
| | (adhE-Ctag in pS453) | solate from S494 |

Expression of Either ACS2 or adhE Increases Acetate Tolerance

The resistance of the strains to acetate was tested on solid YPD media. Initially, the ability of Thermosacc® dry to grow on YPD agar plates containing a range of acetate concentrations from 25 mM (0.14% vol/vol) to 175 mM (1% vol/vol) was tested. These were not adjusted for pH, but all plates were within pH 4 to 4.5. It was observed that Thermosacc® dry failed to grow at 100 mM acetate. Cell growth, assessed by the number and average size of colonies on the plate, was inhibited at 75 mM when compared to growth without acetate. Slight inhibition is observed at 50 mM acetate.

Based on the above findings, a spot assay was conducted for acetate sensitivity of strains 5491 to S495 to test whether the ACS2 and adhE genes would increase acetate resistance. This assay was adapted from the method described by Mollapour et al. (2007; Mol. Cell. Biol. 27 6446-6456) with the following modifications.

Overnight cultures in YPD with clonNAT™ and G418 (OD600 of 5) were serially diluted 10-fold in a microtitre plate. 1.2 μl or 2 μl of each dilution was spotted in series on YPD agar plates containing antibiotics clonNAT™ (100 μg/ml) and G418 (200 μg/ml), inducers CuSO4 (250 μM) and doxycycline (5 μg/ml) and acetate ranging from 0 to 100 mM.

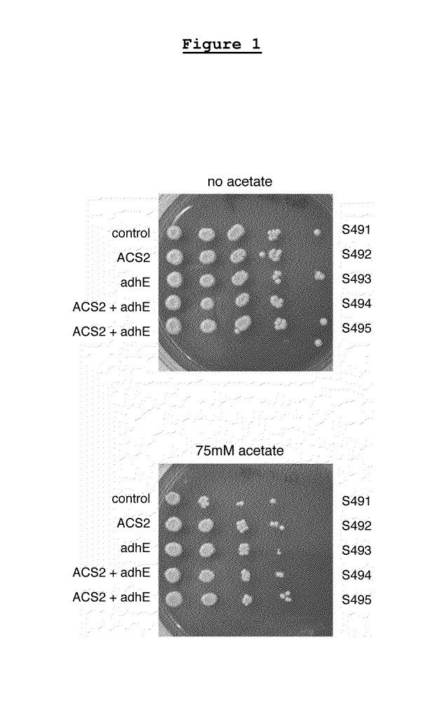

Although all strains grew well without acetate, strains containing adhE and/or ACS2 (S492 to 5495) showed increased cell survival (assessed by the number of colonies on the plate) on 75 mM acetate (FIG. 1) compared to the control strain. The same trend was observed on 80 mM and 90 mM acetate plates; on 100 mM acetate, all strains failed to grow (data not shown).

Expression of Either ACS2 or adhE Increases Initial Growth in Liquid Cultures with 75 mM Acetate

Growth, glucose consumption and ethanol production were measured in the strains grown in YPD with two different concentrations of acetate under conditions of limited oxygen. Optical density was used as a measure of growth, with HPLC used to measure glucose and ethanol in the 1-day old culture media.

15 μl of overnight cultures (grown with just antibiotics and no inducers, OD₆₀₀ of 5) were added into 1.5 ml fresh YPD media containing antibiotics clonNAT™ (100 μg/ml) and G418 (200 μg/ml), inducers CuSO₄ (250 μM) and doxycycline (5 μg/ml) and either 50 mM or 75 mM acetate. They were grown in 2 ml screw cap microfuge tubes (to limit available oxygen) at 30° C. and shaking at 250 rpm. Samples were collected after 24 hours for HPLC and measurement of optical density. Each experiment was done in duplicate.

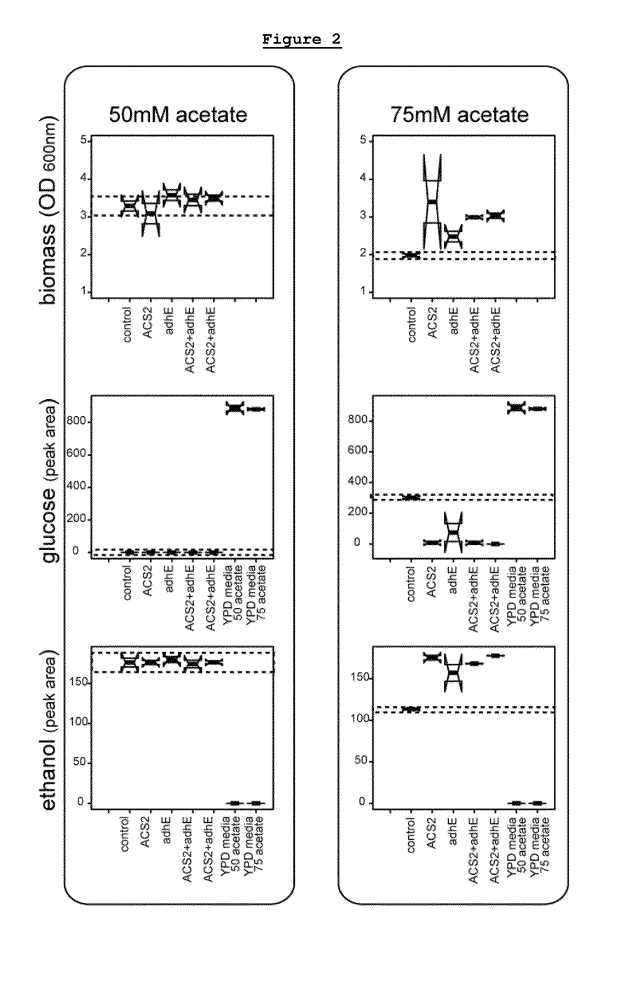

It was determined that all strains grew similarly after 24 hours at 50 mM acetate. All glucose was consumed in all cultures and there were no significant differences in ethanol yield (FIG. 2).

At 75 mM acetate, differences were observed between the control strain and the strains expressing ACS2 and/or adhE (FIG. 2). The control strain grew slowly, reaching an average optical density after 24 hours of 1.98 or 60% of the biomass achieved when grown at 50 mM acetate. Expression of ACS2 led to 71% improved growth over 24 hours compared to control cells expressing only endogenous ACS2, adhE led to 25% improved growth, whilst the two strains expressing both genes grew 51% and 53% better than the control strain.

Increased glucose consumption and ethanol production was also observed in the ACS2 and/or adhE expressing strains (FIG. 2). The wider error ranges for ACS2 (biomass) and adhE (glucose and ethanol) are due to one replicate growing better than the second, however, all replicates showed significantly increased growth and ethanol yield compared to the control strain. The strains expressing both genes, 5494 and 5495, consistently showed around 50% improved ethanol yield after 24 hours over the control strain.

The ACS2 results are consistent with the improved growth observed in sake yeast strains overexpressing ACS2 (Akamatsu et al. (2000) Journal of Bioscience and Bioengineering 90 555-560), although the increased ethanol yield observed herein is, surprisingly, much higher. Piromyces adhE had not previously been expressed in S. cerevisiae and the results indicate that the protein is active in this host.

### Candida ICL1 Promoter Driven Gene Expression

In the work described above, the copper-inducible CUP1 and doxycycline-inducible tetO2 promoters were used to drive expression of adhE and ACS2, respectively. As described above, expression of these genes, either individually or together, increased the growth rate of the yeast Thermosacc® dry strain in the presence of 75 mM acetate (acetic acid in rich YPD medium).

The genes were then expressed under the Candida ICL1 promoter, which is reported to be induced by acetate and ethanol and also at stationary phase when a fermentable carbon source such as glucose has been exhausted (Umumera et al. (1995) Biochim. Et Biophys. Acta 1350 80-88).

Gene expression was initially tested on plasmids. Two new plasmids were constructed in Thermosacc® dry, each bearing either ACS2 or adhE control of the Candida ICL1 promoter. The adhE strain and plasmid were created first by replacement of the CUP1 promoter in pS488 with the Candida ICL1 promoter using gap repair. A DNA sequence (SEQ ID NO:45) containing the Candida ICL1 promoter was designed with about 90 bp of its 5′ sequence homologous to the pS488 vector backbone and 80 bp 3′ sequence homologous to the start of the adhE gene and synthesized (GeneArt). This was co-transformed with

XhoI-digested pS488 into Thermosacc® dry. The resulting clonNAT-resistant transformants (strain 5528 in Table 6) contained plasmid pS538 with ICL1 promoter-driven adhE expression. This was verified by colony PCR using two sets of primers, T7 and Icl1p-216-R (Table 8) to check for plasmid recombination at the 5′ end and primers Icl1p-456-F and adhE-R (Table 8) to check for recombination at the 3′ end.

TABLE 6
Expression of Heterologous Genes from Plasmids

| Yeast | | | |
|---|---|---|---|
| Strain | | | Induction |
| Number | Genotype | Comment | conditions |
| S485 | Thermosacc ® dry + | control strain | 24 hour cultures of: |
| | pS452 [natMX] | | YPD with 1% glucose |
| S564 | Thermosacc ® dry + | producing | YPD with 1% glucose + |
| | pS568 [Icl1p: ACS2- | ACS2 only | 75 mM acetate, |
| | Nstrep, natMX] | | YPD with 1% glucose + |
| | | | 2% ethanol |
| S528 | Thermosacc ® dry + | producing | |
| | pS538 [Icl1p::AdhE- | adhE only | |
| | Cstrep, natMX] | | |

The ACS2 strain and plasmid, bearing an N-terminal strep tag and driven by the Candida ICL1 promoter, was created from pS538 bp gap repair. The strep-tagged ACS2 DNA sequence, with an additional 50 bp DNA homologous to the ICL1 promoter at its 5′ end and an additional 50 bp DNA homologous to the CYC1 terminator at its 3′ end, was created in two rounds of PCR. Using pS453-ACS2 as template, primers strep-ACS2-F and CycT-ACS2-R were used in the first PCR to amplify ACS2 while adding N-terminal strep tag and an overlapping sequence to the CYC1 terminator. This PCR product was used as template for the second PCR using primers Iclp-strep extender and CycT-ACS2-R to add 50 bp of DNA sequence homologous to the Icl1 promoter at the 5′ end. The PCR product was co-transformed with pre-digested pS538 (digested with NdeI and BamHI) into Thermosacc® dry. The clonNAT-resistant transformants (strain 5564 in Table 6) contained plasmid pS568, in which the tagged ACS2 gene is under control of the Candida ICL1 promoter. This was verified by colony PCR using primers ACS2-1988-F and T3 (Table 8).

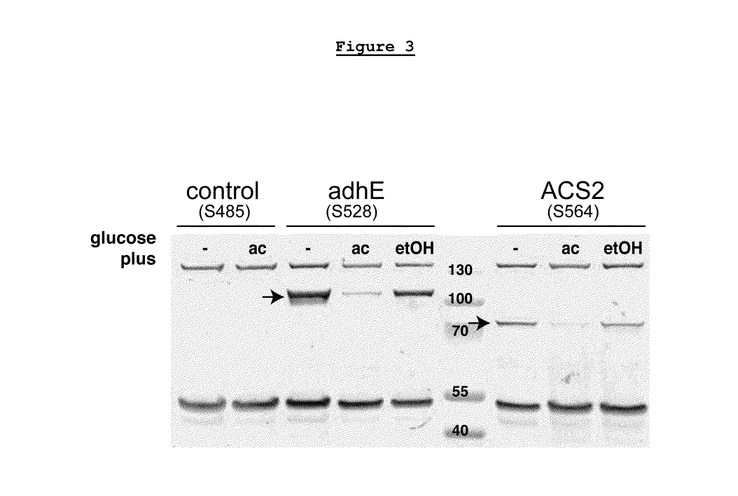

Using Western blots, the production of both tagged proteins was determined after 24 hours in three conditions: YP media (1% yeast extract, 2% bacto peptone) with 1% glucose, YP media supplemented with 1% glucose and 75 mM acetic acid, and YP media with 1% glucose and 2% ethanol (FIG. 3). Proteins were seen in all three conditions.

For the glucose only condition, all cultures reached an optical density at 600 nm (OD₆₀₀) of 10 to 11, which is stationary phase. This explains the strong expression seen of both genes, as the Candida ICL1 promoter has been shown to be active under conditions when a fermentable carbon source such as glucose has been exhausted. The glucose plus ethanol cultures were also in stationary phase, reaching OD₆₀₀ of between 8 and 9, showing good amounts of the tagged proteins. For the glucose and acetate cultures, acetate led to growth inhibition, these cultures had not reached stationary phase and had OD₆₀₀ of between 7 and 8.3. There is less protein visible compared to the other culture conditions, which may be attributed to the presence of glucose that had not been exhausted.

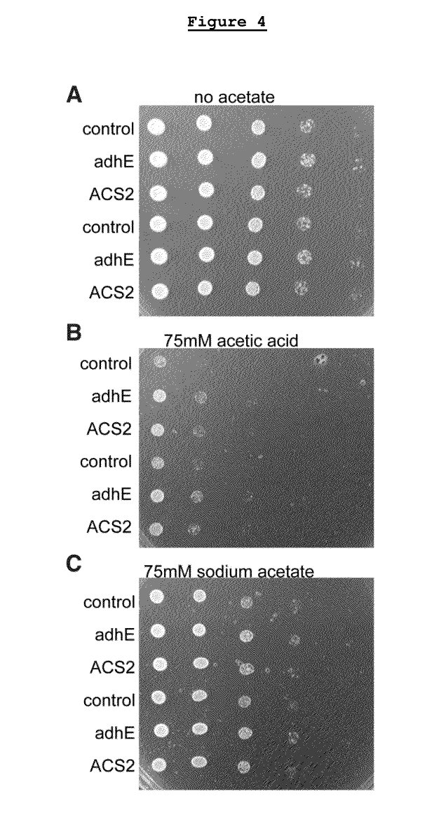

The engineered strains were next assessed for growth on acetate-containing solid media relative to the control strain. The engineered strains also showed increased tolerance to 75 mM acetate, added as either acetic acid or sodium acetate on spot assays (FIG. 4). Overnight cultures in YPD with clonNAT™ (OD₆₀₀ of 5) were serially diluted 10-fold in a microtitre plate. 411 of each dilution was spotted in series on YPD agar plates containing antibiotics clonNAT™ (100 μg/ml) with or without 75 mM acetate. No additional inducers were added to the media and growth was monitored after 24 hours at 30° C.

Thermosacc® dry shows greater sensitivity to acetic acid than sodium acetate, even though both media were adjusted to a pH of 4.5 (FIGS. 4B and 4C). This may be due to more protonated ions in the acetic acid-containing media, to which the yeast cells are sensitive (Taherzadeh et al (1997) Chem. Eng. Sci. 52 2653-2659). On 75 mM acetic acid, adhE and ACS2 led to increased growth after 24 hours at 30° C. (1st and 2nd dilution columns, FIG. 4B). On 75 mM sodium acetate, adhE led to slightly increased growth (3rd and 4th dilution columns, FIG. 4C).

Based on these results, it was concluded that the new plasmids with the Candida ICL promoter led to the production of functional proteins.

Chromosomal Integration of ACS2 and adhE

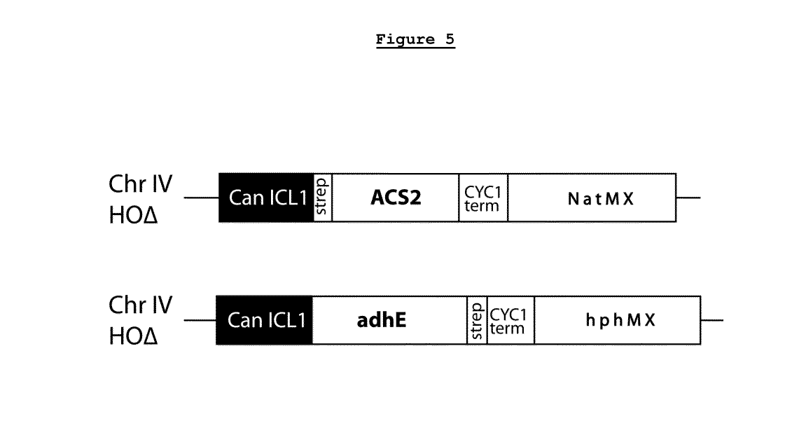

From the above plasmids, two linear DNA constructs were created for the integration of ACS2 and adhE into the HO gene of Thermosacc® dry (FIG. 5). Strains that have integrated the ACS2 construct can be selected with the antibiotic clonNAT™ (nourseothricin; Werner Bioagents) and those that have integrated adhE can be selected with the antibiotic hygromycin B (Invitrogen).

Prior to choosing the integration site, it was confirmed that the genomic sequence that the HO locus is present in Thermosacc® dry and the HO gene has the same mutation as the S288C lab strain that makes it non-functional. In lab strains, deletion of this gene has previously been shown not to affect growth rate or metabolism (Baganz et al. (1997) Yeast 13 1563-1573).

The integration constructs have 100 bp of homology to the flanking sequences of the HO gene. Similar constructs have previously been used by the inventors to target heterologous genes into this locus in lab strains. It was planned to replace the two HO loci in the diploid Thermosacc® dry with one copy of ACS2 and one copy of adhE.

After the first rounds of yeast transformation using the high efficiency lithium acetate transformation protocol (Gietz et al. (2007) Nature Protocols 2 31-34), it was discovered that gene integration in Thermosacc® dry was less efficient than in the S288C-based lab strains. In addition, there were difficulties modifying the adhE construct using conventional cloning techniques due to the poor plasmid yields from E. coli of most plasmids containing this gene.

Through the first two rounds of transformation, two integrants of the ACS2 gene were isolated (strains 5587 and 5588; Table 7). Initially, there were no colonies from adhE or from the co-transformation of the ACS2 and adhE constructs. In comparison, using the same method and DNA concentrations, typically 20-50 integrants were obtained using S288C-based lab strains.

Characterisation of New Thermosacc® Dry Strains with an Integrated Copy of ACS2

For strains 5587 and 5588, integration of ACS2 into one copy of the HO gene was confirmed by colony PCR, leaving the second copy undisrupted (data not shown).

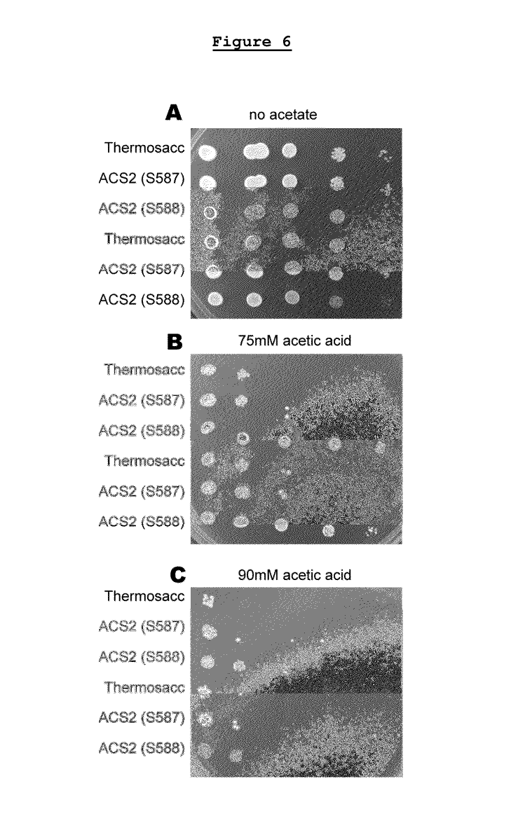

It was observed that these two strains behave differently. Using spot assays, it was observed that both strains show higher tolerance to acetate compared to Thermosacc® dry (FIGS. 6B and 6C). However, 5588 is the more acetate-tolerant of the two strains, showing around 100-fold more resistance than the unmodified Thermosacc® dry, as the 100-fold dilution of 5588 grows as well as undiluted Thermosacc® dry on 75 mM acetic acid (FIG. 6B).

On YPD alone, 5588 grows more slowly than the unmodified strain. This is believed to be due to higher levels of the Acs2 protein, whose overproduction leads to slower growth rate (Yoshikawa et al. (2011) Yeast 28 349-361). This may be due to overproduction of acetyl-CoA in the nucleus that leads to increased acetylation of histones and affecting global gene expression (Galdieri and Vancura (2012) J. Biol. Chem. 287 23865-23876). It is possible that the ACS2 copy integrated into 5588 is more transcriptionally active (than that of S587) because its chromatin structure has altered histone acetylation. Alternatively, it is possible that there is at least one additional copy of ACS2 integrated elsewhere in the genome of 5588.

These phenotypes suggest the integration of a functional ACS2 cassette into chromosome IV of Thermosacc® dry.

Isolation of New Thermosacc® Dry Strains with Integrated Copies of ACS2 and adhE

It was planned to introduce adhE into the second chromosome IV of the ACS2-integrated strains, S587 and S588, selecting on both clonNAT™ and hygromycin in order to construct the double integration strains. It was also planned to introduce adhE into one chromosome of Thermosacc® dry to obtain a single integration control strain.

In order to increase the homologous recombination frequency of the adhE integration construct, transformations were repeated with the original integration construct, this time using 5-fold more DNA (1.5 μg per transformation compared to 300 ng used previously). The original 5.5 kb adhE construct (in FIG. 5) was amplified using the high fidelity Q5 polymerase (New England Biolabs). Using 1.5 μg of DNA resulted in about 50 hygromycin-resistant transformants for each of the three transformations (Thermosacc® dry, S656 and S657).

72 total hygromycin-resistant colonies were screened from all the transformations with Thermosacc® dry (24 screened), 5587 (32 screened) and 5588 (16 screened) strains for correct integration at the HO locus using colony PCR (primers used in Table 8 below). Of these, only two strains had the adhE constructs integrated at the HO locus, these were from the 5588 transformations (S656 and 5657, Table 7). The remaining transformants had random integration, which does not ensure that the complete construct, instead of just the antibiotic resistance cassette, was recombined in the genome.

TABLE 7
Engineered Strains with Integrated Copies of Ic11p-driven Genes

| Yeast | | | |
|---|---|---|---|
| Strain | | | |
| Number | Genotype | Comment | Phenotype |
| S587 | hoΔ::Icl1p- | producing | slight increase in |
| | ACS2::natMX4/HO | ACS2 only | acetate tolerance on |
| | | | YPD at pH 4.5 but not |
| | | | pH 6.5 (FIGS. 5 and |
| | | | 6) |
| S588 | hoΔ::Icl1p- | producing | increase in acetate |
| | ACS2::natMX4/HO | ACS2 only | tolerance on YPD at |
| | | | pH 4.5 but not pH 6.5 |
| | | | (FIGS. 5 and 6) |
| S656 | hoΔ::Icl1p- | producing | increase in acetate |
| | ACS2::natMX4/ | both proteins | tolerance on YPD at |
| | hoΔ::Icl1p- | | pH 4.5 but not pH 6.5 |
| | adhE::hphMX4 | | (FIG. 6) |
| S657 | hoΔ::Icl1p- | producing | (increase in acetate |
| | ACS2::natMX4/ | both proteins; | tolerance on YPD at |
| | hoΔ::Icl1p- | independent | pH 4.5 but not pH 6.5 |
| | adhE::hphMX4 | isolate from S656 | (FIG. 6) |

Characterisation of New Thermosacc® Dry Strains with an Integrated Copy of ACS2 and adhE

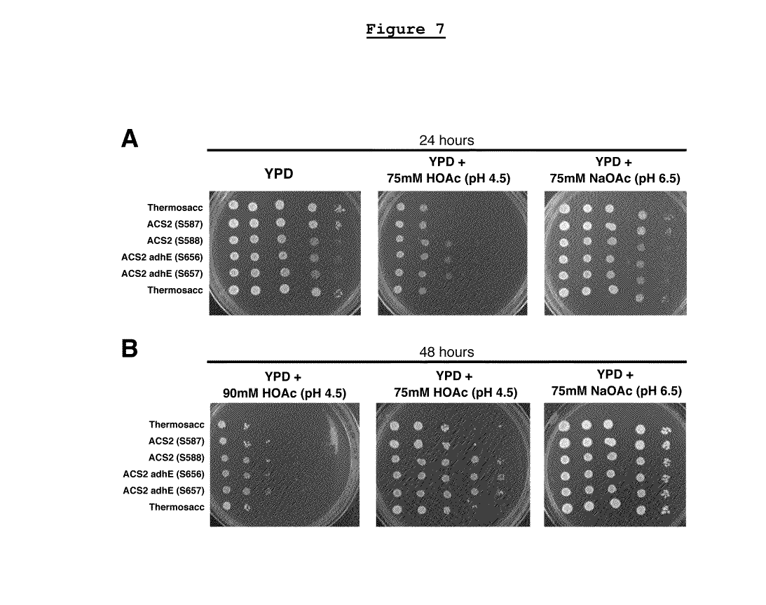

The new double integration strains, 5656 and 5657, containing both ACS2 and adhE, were tested for growth on YPD plates (with 2% glucose) with and without acetate. Growth was compared to Thermosacc® dry and the ACS2-containing strains 5587 and 5588 (FIG. 7).

It was discovered that, like the 5588 ACS2-bearing strain, the new strains were slow-growing compared to Thermosacc® dry (FIG. 7A, YPD). The same number of colonies arose in all engineered strains relative to Thermosacc® dry. However, the 5588, 5656 and 5657 colonies were smaller. These two observations indicate a slower growth rate but the same viability (survival rate) in the engineered strains as Thermosacc® dry on YPD medium.

The same pattern was observed in plates grown in YPD supplemented with 75 mM sodium acetate, with the 5588, 5656 and 5657 strains growing more slowly than Thermosacc® dry. Thermosacc® dry is only slightly sensitive to this medium. The same number of colonies grew on 75 mM sodium acetate as YPD, but the colonies appear just slightly smaller (FIG. 7A, compare 2 rightmost lanes for YPD and YPD+75 mM NaOAc). Unlike the experiment in FIG. 4C, the pH of this medium was not adjusted and it was measured to be around 6.5. Therefore, sodium acetate at near-neutral pH leads to slower growth but no cell death.

On plates containing 75 mM acetic acid (at pH 4.5), sensitivity of the Thermosacc® dry strain was observed. There is approximately a 1% survival rate when compared to growth on YPD, with 100-fold less cell survival (same number of colonies observed in the second dilution or 3rd column on 75 mM HOAc plate [FIG. 7B] as the fourth dilution or 5th column on YPD plate [FIG. 7A]). In contrast, although the engineered strains S588, S656 and S657 were very slow growing on this medium (compare YPD and 75 mM HOAc plates at 24 hours in FIG. 6A), a near 100% survival rate was observed and colonies continued to grow (75 mM HOAc plate at 48 hours in FIG. 7B shows more growth and same number of colonies as YPD plate at 24 hours in FIG. 7A).

Very slow growth was also observed on 75 mM acetic acid (growth rate of individual colonies) for all colonies of all strains. The results above suggest that the overexpression of ACS2 in strain 5588 is sufficient to confer full culture survival on 75 mM acetic acid, but does not increase the growth rate of all surviving yeast cells. Furthermore, addition of adhE (in strains 5656 and 5657) does not lead to any improvement in growth above that conferred by overexpression of ACS2 alone (in S588).

All the integration strains were also tested for growth on 90 mM acetic acid, pH 4.5 (FIG. 7B, left-most plate). A 0.1% survival rate of the Thermosacc® dry strain was observed relative to YPD (FIG. 7A, left-most plate). The engineered strains 5588, 5656 and 5657 improved the survival rate by about 10-fold to 1%. This result is consistent with the observations that the engineered strains are more resistant to the effect of acetic acid. In particular, they have greater cell survival. However, the growth rate or doubling time of surviving cells in acetic acid remains slow.

### Analysis of Strains in Synthetic Complete Media

Acetate depletion cannot be determined by HPLC in YPD media, as a peak from a component of the media overlaps with the acetate peak. Therefore, in order to assess acetate utilisation and ethanol production of the engineered strains using HPLC, two growth experiments were performed in Synthetic Complete medium (SC containing 6.7 g/L yeast nitrogen base with ammonium sulfate, 1.55 g/L complete amino acids, 10 mg/L uracil) with two different carbon source conditions. To measure metabolites using HPLC analysis, supernatants from cultures were run for 30 min on a REZEX ROA Organic Acid column (Phenomenex; 300×7.8 mm, 60° C.; mobile phase: 0.005 N H₂SO₄).

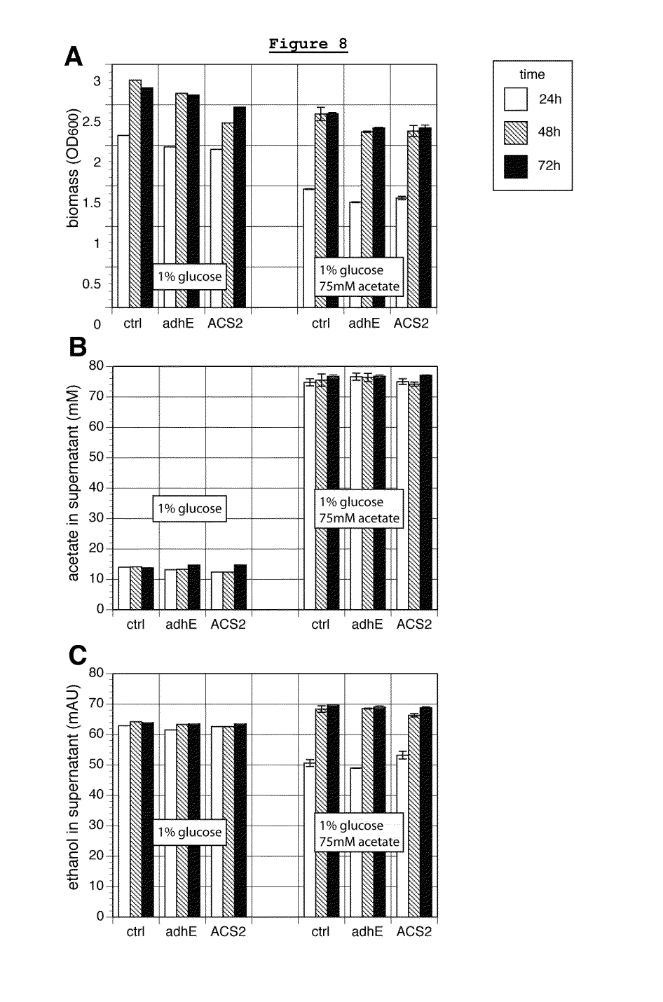

In the first set of experiments, the plasmid-bearing strains adhE (S528) and ACS2 (S564) were compared to the control strain with an empty vector (S485). Growth in media containing 1% glucose was compared to that in media containing 1% glucose and 75 mM acetic acid, which had a pH of around 3.5 (FIG. 8).

In SC with 1% glucose, it was discovered that both the adhE and ACS2 plasmid-bearing strains produced slightly less biomass over three days than the control strain. This pattern was also repeated when strains were grown in SC with 1% glucose and 75 mM acetate (FIG. 8A); although all strains had lower biomass in the presence of acetate, the control strain grew slightly better than the engineered strains. Therefore, expression of adhE or ACS2 from plasmids in SC medium gives a slight growth disadvantage that appears to be independent of acetate. This is in contrast to the findings when these same strains are grown on YPD plates containing 2% glucose and 75 mM acetate (FIG. 4), where expression of adhE or ACS2 leads to more growth in the presence of acetate.

The lower growth rate of the engineered strains may be due to the additional metabolic burden due to overexpression of adhE or ACS2. This may not be observable in rich medium such as YPD, but a nutrient-limited medium such as SC (with half the amount of glucose) may unmask this effect.

Acetate remaining in the medium at 24, 48 and 72 hours after inoculation was also measured (FIG. 8B). When strains were given 1% glucose as the sole carbon source, around 14 mM acetate was detected in the medium at all time-points for all strains, indicating that all strains produced acetate. In medium containing both glucose and 75 mM acetate, no change was detected in the level of acetate in the culture supernatant after 3 days, likely because acetate utilisation is masked by acetate production from all three strains.

Ethanol produced by all strains at 24, 48 and 72 hours was also measured after inoculation (FIG. 8C). No difference was found in ethanol produced when 1% glucose was used as a sole carbon source. When both 1% glucose and 75 mM acetate were present, ethanol production was lower after 24 hours compared to the cultures without acetate. This is consistent with slower biomass production observed in FIG. 8A. At 48 and 72 hours, all acetate-supplemented cultures showed higher levels of ethanol than the cultures with glucose alone (about 10% increase), suggesting that the addition of acetate led to increased ethanol yield. However, no differences in ethanol production were found between the engineered strains and the control strain.

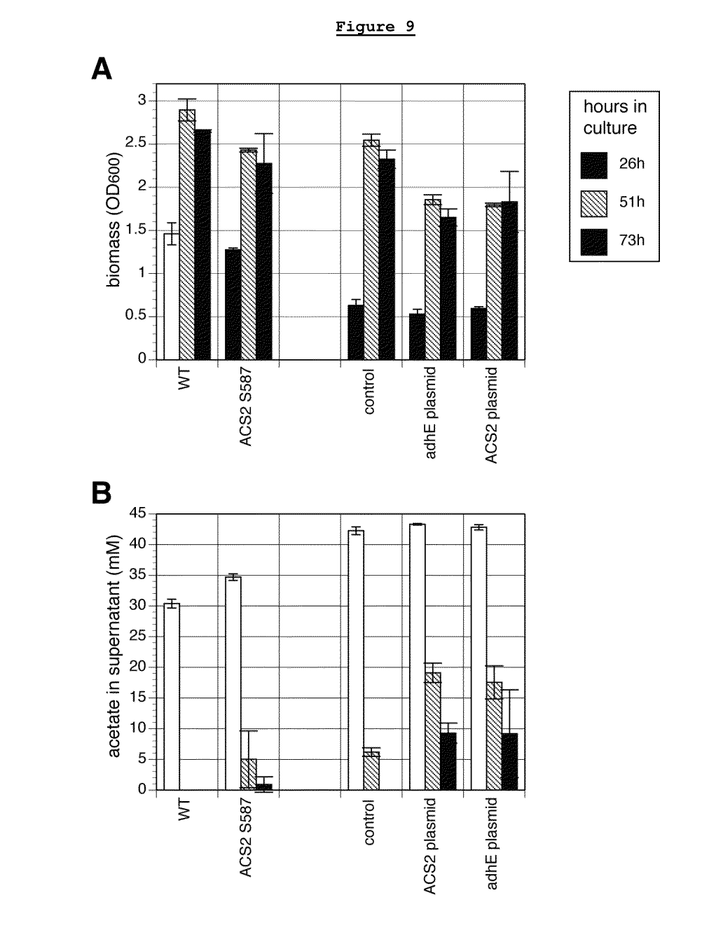

Because the yeast strains produce acetate from glucose, this interfered with the measurement of acetate utilisation (FIG. 9B). Therefore, a second set of experiments was completed in which 50 mM sodium acetate was added to SC medium as a sole carbon source (FIG. 9). At the time this experiment was performed, the effect of pH on the growth of the strains on acetate-containing plates had not been tested (in FIG. 8), so the pH of the SC medium, which was at 7, was not adjusted.

These experiments were performed on both the ACS2 integration strains (S587 and 5588) and compared to the Thermosacc® dry strain (left side of FIGS. 9A and 9B). At the time when this experiment was performed, strains S656 and 5657 had not been generated. Unfortunately, the S588 strain did not grow under these conditions and it is predicted that this strain cannot grow with acetate as a sole carbon source because of too much acetyl-CoA, as discussed above. Therefore, only 5587 was tested. These experiments were also performed on the plasmid strains bearing adhE (S528) and ACS2 (S564) and the control empty vector strain (S485); see right side of FIGS. 9A and 9B.

It was found that all the engineered strains, whether genes were expressed from plasmids or by genome integration, grew more slowly than the control strains, attaining less biomass after 51 and 72 hours of culture (FIG. 9A). Consistent with this, it was also found that less acetate was utilised by the engineered strains, with more acetate remaining in the culture medium at 51 and 72 hours relative to the control strains (FIG. 9B).

These results are consistent with the results on YPD plates containing sodium acetate at neutral pH (FIG. 8), when most of the acetate is deprotonated and does not significantly affect the growth of Thermosacc® dry.

### SUMMARY

In the work presented herein, the inventors have engineered ten Thermosacc® dry-based strains expressing an extra copy of ACS2 and/or adhE (Tables 5, 6 and 7).

These new strains showed more resistance to acetate in YPD medium (at pH 4.5) relative to the Thermosacc® dry control strains. Using spot assays on agar plates, it was determined that inhibitory growth effect of acetate on Thermosacc® dry is due to a reduction in culture survival (reduced number of surviving colonies) as well as a slowing down of cell doubling time (colonies attain the same size 24 hours later relative to colonies on YPD alone). At 75 mM acetic acid on YPD plates, Thermosacc® dry culture shows 1% culture survival; at 90 mM acetic acid, it is 0.1%.

The plate assays also allowed determination that the resistance that ACS2 and/or adhE confer is due to increasing culture survival (from approximately 1% to 100% at 75 mM acetic acid; from approximately 0.1% to 1% at 90 mM acetic acid) but not due to increasing cell-doubling time.

In YPD liquid cultures with 75 mM acetic acid grown for twenty-four hours, the plasmid-based strains grew better and produced more ethanol compared to control strains.

TABLE 8
Primers Used for Engineering Strains

| Sequence | Primer Name | Comment |
|---|---|---|
| CCCTGCAGTGATCATTTC | PstBc1ACS2-R | used to amplify S. cerevisiae ACS2 while |
| TTTTTTTGAGAGAAAAAT | | adding restriction sites for cloning |
| TGGTTC | | |
| (SEQ ID NO: 25) | | |
| CACGCGGCCGCCATGACA | NotACS2-F | |
| ATCAAGGAACATAAAGT | | |
| AG (SEQ ID NO: 26) | | |
| GACATTAACCTATAAAAA | CM251-sw-F | used to amplify kanMX gene and add |
| TAGGCGTATCACGAGGCC | | regions of homology to pCM251 to create |
| CTTTCGTCTTCAAGAATT | | the plasmids pS453 and pS453-ACS2 by |
| GCGCCAGATCTGTTTAGC | | gap repair |
| TTG | | |
| (SEQ ID NO: 27) | | |
| GTTATTTTACAGATTTTAT | CM251-sw-R | |
| GTTTAGATCTTTTATGCTT | | |
| GCTTTTCAAAAGGCTTGC | | |
| AAATTCGAGCTCGTTTTC | | |
| GACA | | |
| (SEQ ID NO: 28) | | |
| CTGGGTGAGCAAAAACA | CM251-gap-F | used to verify kanMX gap repair into |
| GGA | | pCM251-based plasmid; product is 553 bp |
| (SEQ ID NO: 29) | | |
| GACAATTCAACGCGTCTG | pTEF-R | |
| TG | | |
| (SEQ ID NO: 30) | | |
| GGCTTAACTATGCGGCAT | pRSnat-switch-F | used to amplify natMX gene and add |
| CAGAGCAGATTGTACTGA | | regions of homology to pRS416 to create |
| GAGTGCACCATATCGAGC | | |
| GCCAGATCTGTTTAGCTT | | |
| (SEQ ID NO: 31) | | |
| CGTTTACAATTTCCTGAT | pRSnat-switch-R | |
| GCGGTATTTTCTCCTTAC | | |
| GCATCTGTGCGGTATTTC | | |
| AATTCGAGCTCGTTTTCG | | |
| AC | | |
| (SEQ ID NO: 32) | | |
| CGTTGGAGTCCACGTTCT | pRS-check-sw-R | used to verify natMX gap repair into pRS- |
| TT | | based plasmid; PCR product is 477 bp |
| (SEQ ID NO: 33) | | |
| GGATGGGGTTCACCCTCT | natC | |
| G | | |
| (SEQ ID NO: 12) | | |
| CCGGACGACAGAGCATAT | adhE-Nde-Cstrep- | used to amplify AdhE with C-terminal tag |
| GAAAAATGTCCGGTTTAC | F | |
| AAATGTTCC | | |
| (SEQ ID NO: 34) | | |
| TCGGATCCTCACTTTTCG | adhE-Cstrep-Bam | used to amplify AdhE with C-terminal tag |
| AATTGTGGGTGAGACCAt | R | |
| CCACCCCCGCCTCCCCCC | | |
| AACTTTGGAACAACACCA | | |
| TTCCA | | |
| (SEQ ID NO: 35) | | |
| ACAAACTGTACAATCAAT | CupP-extender-F | used to amplify AdhE with C-terminal tag |
| CAATCAATCATCACATAA | | |
| ATCCGGACGACAGAGCAT | | |
| ATG | | |
| (SEQ ID NO: 36) | | |
| GCGGATGTGGGGGGAGG | CycT-extender-R | used to amplify AdhE with C-terminal tag |
| GCGTGAATGTAAGCGTGA | | |
| CATAACTAATTACATGAC | | |
| TCGGATCCTCACTTTTCG | | |
| AA | | |
| (SEQ ID NO: 37) | | |
| CTACAAGGCCAAGGATTT | adhE-1077-F | used to amplify AdhE with C-terminal tag |
| CG | | |
| (SEQ ID NO: 38) | | |
| GACGGACTTTGGGATCTT | adhE-1425-R | used to amplify AdhE with C-terminal tag |
| GA | | |
| (SEQ ID NO: 39) | | |
| TAATACGACTCACTATAG | T7 | used to verify gap repair of C-terminal |
| GG | | tagged AdhE into pS446; PCR product is |
| (SEQ ID NO: 6) | | 752 bp |
| TGAGTTTCTTCATGGGCG | adhE-R | |
| TA | | |
| (SEQ ID NO: 9) | | |
| AACCACACGCACACGACT | ICL1p-216-R | used to verify gap repair of ICL1 |
| CT | | promoter; 278 bp PCR product produced in |
| (SEQ ID NO: 10) | | combination with primer T7 |
| GCAGTTTTCCCTTTCCTCC | ICL1p-456-F | used to verify gap repair of ICL1 promoter; |
| A | | 346 bp PCR product produced in |
| (SEQ ID NO: 40) | | combination with primer adhE-R |
| ATGGGTTGGAGCCACCCG | strep-ACS2-F | used for the first round of PCR to amplify |
| CAGTTCGAAAAAGGCGG | | ACS2 while adding N-terminal strep tag; |
| AGGTATGACAATCAAGG | | used in combination with CycT-ACS2-R |
| AACATAAAGTAG | | |
| (SEQ ID NO: 41) | | |
| GCGGATGTGGGGGGAGG | CycT-ACS2-R | used for PCR to amplify ACS2 while |
| GCGTGAATGTAAGCGTGA | | adding a 3′ sequence homologous to the |
| CATAACTAATTACATGAC | | Cyc1 terminator; used in combination with |
| TCggatccTCATTTCTTTTTT | | strep-ACS2-F for first PCR amplification |
| TGAGAGAAAAATTGGTTC | | and Ic1p-strep extender for second PCR |
| (SEQ ID NO: 42) | | amplification |
| CATCCTTTATAATTGTCTA | Ic1p-strep extender | used for the second round of PCR to |
| ACCAACAACTATATATCT | | amplify ACS2 with the N-terminal strep |
| ATCAACCATATGAAAAAT | | tag and add homology to the Icl1 promoter; |
| GGGTTGGAGCCACCCGCA | | used in combination with CycT-ACS2-R |
| G | | |
| (SEQ ID NO: 43) | | |
| TACTTTGGCCAACCCAGA | ACS2-1988-F | used to verify gap repair of ACS2 in |
| AG | | plasmid pS468; PCR product is 469 bp |
| (SEQ ID NO: 44) | | |
| ATTAACCCTCACTAAAGG | T3 | |
| GA | | |
| (SEQ ID NO: 5) | | |
| CGAATAAAGTCGCGGAA | HO-5-F | Forward primer for use with SEQ ID |
| AAA | | NOs: 8, 9, 10 |
| (SEQ ID NO: 7) | | |
| TCAAGCGTCTGACATTGC | HO-noins-R | Negative control reverse primer producing |
| TG | | 589 bp DNA when there is no integration at |
| (SEQ ID NO: 8) | | HO site |
| TTTTGTTTTCTCTTAACTT | HO-3-R | Reverse primer for use with SEQ ID |
| TGTATCCT | | NOs: 12, 13 |
| (SEQ ID NO: 11) | | |
| ACTCGCCGATAGTGGAAA | hygC | Forward primer producing 635 bp product |
| CC | | when adhE construct is integrated correctly |
| (SEQ ID NO: 13) | | at HO site |

TABLE 9
Plasmids & DNA constructs

| plasmid number | description |
|---|---|
| pS452 | empty vector based on pRS416 (Sikorski and Hieter, 1998); |
| (SEQ ID NO: 16) | pRS415 was modified by replacing the original URA3 marker |
| | with the natMX marker; this was done by gap repair. |
| pS453 | empty vector based on pCM251 (Belli et al., 1998); pCM251 was |
| (SEQ ID NO: 17) | modified by replacing the original TRP1 marker with the kanMX |
| | marker; this was done by gap repair. |
| pS440 | intermediate plasmid used in the construction of pS465 and pS488 |
| (SEQ ID NO: 24) | |
| pS446 | intermediate plasmid used in the construction of pS453-ACS2 |
| (SEQ ID NO: 22) | |
| pS453-ACS2 | S453 with insert N-terminally hexahistidine-tagged ACS2 |
| (SEQ ID NO: 15) | |
| pS465 | S452 with insert N-terminally strep-tagged AdhE flanked by the |
| (SEQ ID NO: 23) | CUP1 promoter and the CYC1 terminator |
| pS488 | S452 with insert C-terminally strep-tagged AdhE flanked by the |
| (SEQ ID NO: 14) | CUP1 promoter and the CYC1 terminator |
| pS538 | S452 with insert C-terminally strep-tagged AdhE flanked by the |
| (SEQ ID NO: 18) | Candida ICL1 promoter and the CYC1 terminator; created by gap |
| | repair from S488 |
| pS568 | S452 with insert N-terminally strep-tagged ACS2 flanked by the |
| (SEQ ID NO: 19) | Candida ICL1 promoter and the CYC1 terminator; created by gap |
| | repair from S528 |
| C18 | adhE integrating construct into HO locus with hygromycin marker |
| (SEQ ID NO: 20) | (represented in FIG. 5 bottom) |
| C19 | ACS2 integrating construct into HO locus with natMX marker |
| (SEQ ID NO: 21) | (represented in FIG. 5 top) |

1. A modified Saccharomyces cerevisiae cell that differs from an unmodified cell in that it comprises an adhE polypeptide having at least 50% sequence identity to the amino acid sequence of SEQ ID NO:1, wherein the adhE polypeptide is capable of catalysing the formation of ethanol from acetyl CoA.

2. The cell of claim 1 having at least 75% sequence identity to the amino acid sequence of SEQ ID NO:1.

3. The cell of claim 1, further comprising increased expression of an ACS2 polypeptide having at least 50% sequence identity to the amino acid sequence of SEQ ID NO:2.

4. The cell of claim 1 having an increased ability to grow on or in a medium containing at least about 50 mM acetate.

5. The cell of claim 1 wherein the unmodified cell is a Thermosacc® dry cell.

6. The cell of claim 1 which has increased ethanol production over at least about 20 hours in comparison to an unmodified cell.

7. The cell of claim 6 wherein the increased ethanol production is an increase of at least about 40%.

8. The cell of claim 1 further comprising a polynucleotide sequence encoding the adhE polypeptide, wherein the polynucleotide sequence is SEQ ID NO:3.

9. The cell of claim 3 further comprising a polynucleotide sequence encoding the ACS2 polypeptide, wherein the polynucleotide sequence is SEQ ID NO:4.

10. A modified Saccharomyces cerevisiae cell that differs from a unmodified cell in that it has increased expression of an ACS2 polypeptide having at least 50% sequence identity to the amino acid sequence of SEQ ID NO:2, wherein the ACS2 polypeptide is capable of catalysing the conversion of acetate to acetyl CoA and wherein the unmodified cell is not the sake yeast strain Kyokai no. 7 or the strain Kyokai no. 701.

11. A method of improving the ability of a yeast cell to grow on a solid medium, or in a liquid medium, the medium comprising at least about 50 mM acetate, wherein the method comprises:
introducing to the yeast cell an adhE polypeptide having at least 50% sequence identity to the amino acid sequence of SEQ ID NO:1, wherein the adhE polypeptide is capable of catalysing the formation of ethanol from acetyl CoA.

12. The method of claim 11 further comprising:
increasing expression of an ACS2 polypeptide having at least 50% sequence identity to the amino acid sequence of SEQ ID NO:2, wherein the ACS2 polypeptide is capable of catalysing the conversion of acetate to acetyl CoA.

13. The method of claim 11 wherein the cell is a S. cerevisiae cell.

14. The method of claim 11 further comprising:
transforming the cell with a polynucleotide encoding the adhE polypeptide, wherein the polynucleotide sequence is SEQ ID NO:3.

15. The method of claim 11 further comprising:
transforming the cell with a polynucleotide encoding the ACS2 polypeptide, wherein the polynucleotide sequence is SEQ ID NO:4.

16. The method of claim 14 wherein the polynucleotide is contained within a vector.

17. The method of claim 14 wherein the polynucleotide becomes integrated into the cell genome

18. The method of claim 11 wherein the improved ability of the yeast cell to grow in a liquid medium comprising at least about 75 mM acetate is at least about 25% increased growth compared to an equivalent cell not comprising the adhE polypeptide.

19. The method of claim 11 wherein the improved ability to grow on a solid medium comprising at least about 75 mM acetate is about 100% cell survival.

20. A method of increasing ethanol yield up to about 30 hours from a yeast cell, comprising:
a. expressing in the yeast cell an adhE polypeptide having at least 50% sequence identity to the amino acid sequence of SEQ ID NO:1, wherein the adhE polypeptide is capable of catalysing the formation of ethanol from acetyl CoA; and
b. culturing the cell in or on a medium comprising glucose.

21. The method of claim 20 further comprising:
over expressing in the yeast cell an ACS2 polypeptide having at least 50% sequence identity to the amino acid sequence of SEQ ID NO:2, wherein the ACS2 polypeptide is capable of catalysing the conversion of acetate to acetyl CoA.
